(19) United States
(12) Patent Application Publication
(10) Pub. No.: US 2022/0001083 A1
(43) Pub. Date: Jan. 6, 2022

(54) METHOD FOR THE MODIFICATION OF A DEVICE SURFACE BY GRAFTING A CD31-DERIVED PEPTIDE ONTO THE SURFACE OF SAID DEVICE

(71) Applicants: BALT EXTRUSION, Montmorency (FR); INSTITUTE NATIONAL DE LA SANTE ET DE LA RECHERCHE MEDICALE (INSERM), Paris (FR); UNIVERSITE PARIS 13 - PARIS NORD, Villetaneuse (FR); UNIVERSITÉ DE PARIS, Paris (FR)

(72) Inventors: Antonino NICOLETTI, Montrouge (FR); Giuseppina CALIGURI, Montrouge (FR)

(21) Appl. No.: 17/296,851

(22) PCT Filed: Nov. 27, 2018
(86) PCT No.: PCT/IB2018/001498

§ 371 Date: May 25, 2021

(51) Int. Cl. A61L 31/02; A61L 31/10; A61L 31/16

(52) U.S. Cl. CPC A61L 31/022; A61L 31/16; A61L 31/10

(57) ABSTRACT

Disclosed is a method for the modification of the surface of a metallic implantable device for interventional neuroradiology by grafting a CD31-derived peptide onto the surface of the device, wherein the CD31-derived peptide consists of a sequence selected from: SEQ ID NO: 2 to 8, SEQ ID NO: 12 to 79, and SEQ ID NO: 81, the method includes: a) coating a polydopamine layer onto the surface of the device in order to obtain a polydopamine coated surface; b) modifying the polydopamine coated surface by adding a linker including at least one reactive moiety chosen from alkyne functions, to obtain a modified polydopamine coated surface; and c) adding a CD31-derived peptide including an azide terminal group and its reaction with the alkyne function of the linker of step b), to obtain a polydopamine coated surface grafted by a CD31-derived peptide.

### CROSS-REFERENCE TO RELATED APPLICATIONS

This application is the U.S. national phase of International Application No. PCT/IB2018/001498 filed Nov. 27, 2018 which designated the U.S., the entire contents of which is hereby incorporated by reference.

### REFERENCE TO SEQUENCE LISTING SUBMITTED ELECTRONICALLY

The content of the electronically submitted sequence listing (Name: 7111-360_SEQLISTING.txt; Size: 40.1 kilobytes; and Date of Creation: May 24, 2021) filed with the application is incorporated herein by reference in its entirety.

### BACKGROUND OF THE INVENTION

### Field of the Invention

The present invention concerns a method for the modification of a device surface, such as metallic implantable devices for interventional neuroradiology, by grafting a CD31-derived peptide onto the surface of said device, as well as the modified surface device obtainable by said method. The present invention also relates to a CD31-specific biomimetic peptide coating and the method for a standardized and oriented grafting of which fosters the attachment and physiologic function of endothelial cells onto endovascular-suitable prosthetic surfaces, as well as the use of the coated devices obtainable by said method.

### Description of the Related Art

Arterial stents are made of an interconnecting network of solid elements, or struts, made of wires, tubes, or sheets, rolled in cylinder shaped scaffolding conceived to maintain adequate blood flow rate and direction in diseased arteries. Their use has become unavoidable in the management of arterial stenosis, across which balloon-inflatable stents are implanted to keep patent the targeted arterial segment and is gaining increasing importance in the management of aneurysms, from which the arterial flow is diverted by auto-expandable, tightly meshed stents, or embolized with intrasaccular implants associated or not with intracranial stents.

Although balloon-expandable stents and flow diverting stents were developed for the treatment of two different arterial pathologies (reopen stenotic arteries to prevent organ ischemia vs diverting the flow from arterial saccular aneurysms to prevent the hemorrhage due to its rupture), and the working mechanisms of these devices is different, both are associated with complications stemming from biocompatibility issues. In particular, the rapid migration, growth to confluence and acquisition of a physiologic (anti-inflammatory and anti-thrombotic) phenotype of the adjacent arterial endothelial cells onto the stent struts is key to the integration and perfect function of the endoprosthesis in the treated arterial segment. No such biocoating exists for flow diverters.

Several strategies aimed at conferring the device with adhesive properties for endothelial cells are already available (capture of circulating endothelial progenitors by anti-CD34 antibodies, use of synthetic peptides derived from the RGD repeated sequence of extracellular matrix components) but none is able to actively and specifically promote endothelial physiologic functions. The latter are critically driven by the engagement of the trans-homophilic CD31 receptor, which is constitutively expressed at a very high density at the surface of endothelial cells (10⁶ molecules/cell).

The surface of devices such as stents does not display the chemical functions required for the conjugation of biomolecules. Thus, their surface must be ‘functionalized’ for the subsequent covalent immobilization of a bioactive molecule such as a peptide. A possible approach that allows the direct immobilization of the peptide on an alloy is the plasma glow discharge. However, the use of this technique on certain metallic surfaces, such the nitinol, is challenging, and alternative polymer-based solutions have to be considered.

The use of polymer coatings as intermediate layers for the immobilization of bioactive molecules has several advantages. The first one is that, unlike almost all other types of materials, most polymers either contain functional groups that can react with bioactive molecules, or are easy to functionalize with such groups. The second one is that polymers are generally inexpensive and easy to process into coatings. Finally, there exists a very broad range of polymers, which allows for the fine-tuning of the chemical properties of the coatings. For these reasons, the use of polymer films is generally considered as an optimal strategy for the immobilization of bioactive molecules and has been the preferred system in the design of coated stents.

However, the most used polymer films do have some limitations, especially in their application as biomaterials coating. Their adhesion to the metal, their resistance to stent deployment, and their stability properties must be adapted to the intended use, in order to prevent the deleterious biological effects of delamination (the detachment of the film from its substrate) and uncontrolled degradation. Above all, the biocompatibility of the polymer coatings and of their degradation products is key to the biological performances of coated stents.

Polymer films can be deposited through a multitude of coating methods. Reproducibility, scalability and limited cost are essential requirements for any process developed for an industrial application, and coating methods for medical devices are no exception. In addition, processes used in the manufacturing of medical devices need to be meet some extra criteria regarding the safety and sterility of the end product, stated by the authorities in the “Good Manufacturing Practices”.

### SUMMARY OF THE INVENTION

The aim of the present invention is thus to provide a method for immobilizing a CD31-derived peptide, in particular a CD31-mimetic peptide on a device surface, in particular a stent surface for interventional neuroradiology, allowing a strong anchoring of said CD31-derived peptide.

The aim of the present invention is also to provide a method for immobilizing a CD31-derived peptide on a device surface, in particular a stent surface, being reproducible, scalable and with limited cost, and also satisfying the requirements regarding the safety and sterility of the end product.

Therefore, the present invention relates to a method for the modification of the surface of a metallic implantable device for interventional neuroradiology by grafting a CD31-derived peptide onto the surface of said device, wherein the CD31-derived peptide consists of a sequence selected from the group consisting of: SEQ ID NO: 2 to 8, SEQ ID NO: 12 to 79, and SEQ ID NO: 81, said method comprising the following steps:

a) the coating of a polydopamine layer onto the surface of a metallic implantable device for interventional neuroradiology in order to obtain a polydopamine coated surface;

b) the modification of the polydopamine coated surface by the addition of a linker, in particular a biorthogonal linker, comprising at least one reactive moiety chosen from alkyne, in particular cyclooctyne, functions, in order to obtain a modified polydopamine coated surface; and

c) the addition of a CD31-derived peptide comprising an azide terminal group and its reaction with the alkyne function of the linker of step b), in order to obtain a polydopamine coated surface grafted by a CD31-derived peptide, wherein the CD31-derived peptide comprising an azide terminal group is a CD31-derived peptide as defined above which is chemically modified with an azide terminal group.

Thus, the present invention provides a method for immobilizing a CD31-derived peptide, in particular a CD31-mimetic peptide on a device surface, in particular a surface of a metallic implantable device for interventional neuroradiology, allowing a strong anchoring of said CD31-derived peptide onto a polydopamine polymer functionalized by biorthogonal copper-free chemistry allowing for a standardized density and controlled orientation of said peptide.

Step a)

As mentioned above, the method of the invention comprises a step consisting in coating a polydopamine layer onto the surface of the device. This step thus leads to a polydopamine coated surface.

The device obtained after step a) corresponds to the starting device the surface of which is coated with a polydopamine layer.

Polydopamine (PDA) is a bio-inspired, self-assembling polymer which was discovered in 2007 by Messersmith and colleagues. This discovery originated from their investigations on the adhesive proteins of marine mussels. Dopamine, a small molecule previously known for its biological role as a neurotransmitter, which combines an amine and a catechol group (which is converted into quinone by oxidation), when dissolved in an aqueous buffer at a slightly basic pH, self-polymerizes into a very adherent film, on various types of substrates. Besides, PDA exhibits latent reactivity towards amine and thiol groups, which makes it a very attractive substrate for bioactive molecule immobilization.

As for the biocompatibility properties of PDA, they appeared to be very adapted to its application as a stent coating application: it was shown to promote endothelial cell adhesion and proliferation, to decrease platelet adhesion and to reduce SMC proliferation (Ku, S. H., Ryu, J., Hong, S. K., Lee, H. and Park, C. B. 2010. Biomaterials 31(9): 2535-2541; Yang, Z., Tu, Q., Zhu, Y., Luo, R., Li, X., Xie, Y., Maitz, M. F., Wang, J. and Huang, N. 2012. Advanced Healthcare Materials 1(5): 548-559; Luo, R., Tang, L., Zhong, S., Yang, Z., Wang, J., Weng, Y., Tu, Q., Jiang, C. and Huang, N. 2013. ACS Applied Materials & Interfaces 5(5): 1704-1714).

According to an embodiment, step a) comprises contacting, under stirring, the surface of the device with an alkaline (preferably at pH 8.5) solution of dopamine in the air and incubating said device and said solution, preferably at a temperature comprised between 18° C. and 30° C., in particular at room temperature, and preferably for a duration comprised between 18 hours and 30 hours, in particular comprised between 20 h and 24 h.

Preferably, step a) is followed by a rinsing step of the polydopamine coated surface, in particular with deionized water.

According to an embodiment, the polydopamine layer has a thickness comprised between 20 nm and 100 nm, preferably between 30 nm and 50 nm, and more preferably of 45 nm.

Step b)

As mentioned above, step b) consists in modifying the polydopamine coated surface of step a) through the fixation of a biorthogonal, copper-free click chemistry-suitable linker. This step thus leads to a modified polydopamine coated surface, which comprises the polydopamine layer as defined above and a layer comprising the linker onto the surface of the device.

The device obtained after step b) corresponds to the starting device comprising a polydopamine layer on its surface, said polydopamine layer being further coated with a layer comprising the linker as defined above.

According to an embodiment, step b) comprises contacting the polydopamine coated surface of the device with a solution of the linker and incubating said device and said solution, under stirring, preferably at a temperature comprised between 18° C. and 30° C., in particular at room temperature, and preferably for a duration comprised between 18 hours and 30 hours, in particular comprised between 20 h and 24 h.

Preferably, step b) is followed by a rinsing step of the modified polydopamine coated surface, in particular with deionized water.

According to an embodiment, the layer made of the linker is obtained has a thickness comprised between 0.03 nm and 3 nm, preferably between 0.1 nm and 0.2 nm, and more preferably of 0.15 nm.

Linker

The linker according to the invention which is used in step b) is an alkyne derivative, and preferably a cyclooctyne derivative, and is thus characterized by the presence of at least one triple bond, especially able to react with an azide group, in particular by click chemistry.

According to an embodiment, the linker has the formula (I-1):

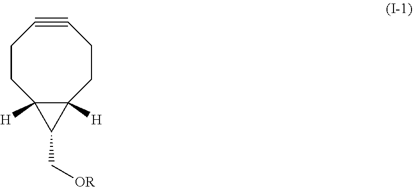

wherein R is a radical of formula —X₁-A₁-NH₂,
X₁ being chosen from the group consisting of: —CONH—, —CO—, —CS— and —CSNH, X₁ being preferably —CONH— or —CO—, and
A1 being an alkylene radical comprising from 2 to 40 carbon atoms, possibly interrupted by at least one oxygen atom.

According to a preferred embodiment, the linker according to the invention comprising at least one alkyne function has the following formula (I):

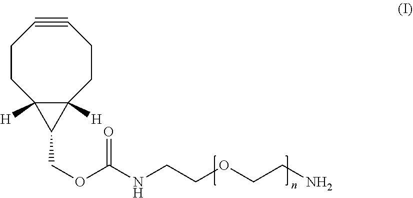

wherein n is an integer comprised between 2 and 14.

Preferably, the linker according to the invention has the following formula (II):

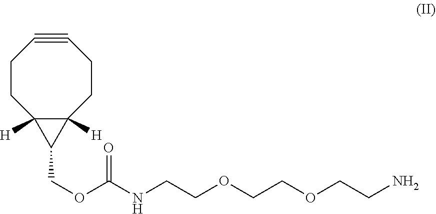 [SMILES: NCCOCCOCCNC(=O)OC[C@@H]1[C@@H]2CCC#CCC[C@@H]21]

Step c)

As mentioned above, step c) consists in further modifying the modified polydopamine coated surface of step b) through the addition of a CD31-derived peptide able to react with the linker as mentioned above. This step thus allows the immobilization or grafting of said CD31-derived peptide onto the device surface.

Step c) thus leads to a modified polydopamine coated surface grafted by a CD31-derived peptide, which comprises the polydopamine layer as defined above and a layer comprising the linker onto the surface of the device as defined above.

The device obtained after step c) corresponds to the starting device as defined above comprising a polydopamine layer on its surface, said polydopamine layer being further coated with a layer made of the linker as defined above and the CD31-derived peptide as defined above onto the surface of the device.

According to an embodiment, step c) comprises contacting the modified polydopamine coated surface of the device with a solution of the CD31-derived peptide comprising an azide terminal group in water, at room temperature during 24 hours, at a concentration comprised between 0.001 μM/cm² and 200 μM/cm² of surface of the device.

Preferably, step c) comprises a step of copper-free click chemistry reaction.

According to an embodiment, the thickness of the layers made of the linker and of the CD31-derived peptide is comprised between 0.5 nm and 15 nm, preferably between 1 nm and 10 nm, and more preferably of 5.6 nm.

According to an embodiment, the thickness of the polydopamine layer and of the layers made of the linker and of the CD31-derived peptide is comprised between 20 nm and 200 nm, preferably between 10 nm and 160 nm, and more preferably between 30 nm and 120 nm.

CD31-Derived Peptide Comprising an Azide Terminal Group

As mentioned above, the CD31-derived peptide to be grafted or immobilized on the device surface comprises an azide terminal group and is thus able to react with the linker comprising at least one triple bond, especially by click chemistry.

The CD31-derived peptide comprising an azide terminal group according to the invention is a CD31-derived peptide as defined below, which is chemically modified with an azide terminal group.

The CD31-derived peptide comprising an azide terminal group according to the invention is a CD31-derived peptide which is chemically modified with an azide terminal group, wherein the CD31-derived peptide consists of a sequence selected from the group consisting of: SEQ ID NO: 2 to 8, SEQ ID NO: 12 to 79, and SEQ ID NO: 81.

The advantages of a CD31 mimetic coating of surfaces such as stents are achieved through the present method of coating stents based on a simple, reproducible and scalable three dip-coating step procedure, wherein the bioactive agent is a retroinverso CD31 mimetic peptide and its coating is density- and orientation-controlled by the use of biorthogonal copper-free chemistry.

According to a preferred embodiment, the CD31-derived peptide comprising an azide terminal group has the formula:

N⁻═N⁺═N—(CD31-derived peptide)-OH

wherein the CD31-derived peptide consists of a sequence selected from the group consisting of: SEQ ID NO: 2 to 8, SEQ ID NO: 12 to 79, and SEQ ID NO: 81.

According to a preferred embodiment, the CD31-derived peptide used in the present invention is a CD31-derived peptide, which is then modified (i) optionally, by adding a spacer at the N-terminus end and (ii) by the grafting of an azide-terminal group.

According to a preferred embodiment, the CD31-derived peptide comprising an azide terminal group has the formula:

N⁻═N⁺═N-(spacer)-(CD31-derived peptide)-OH

wherein:
the spacer is an amino acid spacer comprising at least 2 amino acids, preferably at least 3 amino acids, and
the CD31-derived peptide is as defined below.

The spacer may for example consist of 2 to 14 amino acids, for example 4, 5, 6, 7, 8, 9 or 10 amino acids.

The spacer may for example consist of sequence KGGG (SEQ ID NO: 80), wherein the amino acids are preferably D-enantiomer amino acids.

This embodiment allows the promotion of the regulatory functions of CD31 in the cells that directly enter in contact with the device, such as stent. Thus, the CD31 coating confers anti-thrombotic and anti-inflammatory properties to the surface, and, above all, it promotes the rapid formation of a functional endothelium on the stent struts. The CD31 agonist P8RI peptide as mentioned below is designed to achieve this goal by targeting the CD31 sequence involved in the cis-homophilic engagement which naturally occurs when endothelial cells, leukocytes, or platelets enter in contact with each other, and which is essential for the intracellular CD31 signaling.

CD31-Derived Peptide

As explained above, the CD31-derived peptide (also called CD31 peptide) is used as a peptide (which may also be named initial peptide) and is further chemically modified in order to obtain the CD31-derived peptide comprising an azide terminal group according to the invention.

The initial peptide may be a peptide as disclosed in WO2010/000741 or WO2013/190014 (before its modification with an azide-terminal group and optionally before adding a spacer).

The CD31-derived peptides of the invention are in particular fragments of the sequence of human CD31 (SEQ ID NO: 1) or of murine CD31 (SEQ ID NO: 9).

Other non-limiting examples of non-human mammalian CD31 are the bovine CD31 of sequence SEQ ID NO: 10 and the pig CD31 of sequence SEQ ID NO: 11.

According to the invention, the initial peptide is a CD31-derived peptide as defined above consisting of a sequence selected from the group consisting of: SEQ ID NO: 2 to 8, SEQ ID NO: 12 to 79, and SEQ ID NO: 81.

Therefore, said initial peptide consists of a sequence selected from the group consisting of: SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, and SEQ ID NO: 81.

According to an embodiment, the initial peptide is selected in the group consisting of a peptide of sequence SEQ ID NO: 2, a peptide of sequence SEQ ID NO: 3 (VRVFLAPWKK, amino acids 581 to 590 of SEQ ID NO: 9), a peptide of sequence SEQ ID NO: 4, a peptide of sequence SEQ ID NO: 5, a peptide of sequence SEQ ID NO: 6 consisting of D-enantiomer amino acids, a peptide of sequence SEQ ID NO: 7, a peptide of sequence SEQ ID NO: 8 consisting of D-enantiomer amino acids, and a peptide of sequence SEQ ID NO: 12 (amino acids 579 to 601 of sequence SEQ ID NO: 1).

As mentioned above, the initial peptide has a sequence selected from the group consisting of: SSTLAVRVFLAPWKK (SEQ ID NO: 13, amino acids 576 to 590 of SEQ ID NO: 9), STLAVRVFLAPWKK (SEQ ID NO: 14, amino acids 577 to 590 of SEQ ID NO: 9), TLAVRVFLAPWKK (SEQ ID NO: 15, amino acids 578 to 590 of SEQ ID NO: 9), LAVRVFLAPWKK (SEQ ID NO: 16, amino acids 579 to 590 of SEQ ID NO: 9), AVRVFLAPWKK (SEQ ID NO: 17, amino acids 580 to 590 of SEQ ID NO: 9), VRVFLAPWKK (SEQ ID NO: 3, amino acids 581 to 590 of SEQ ID NO: 9), RVFLAPWKK (SEQ ID NO: 18, amino acids 582 to 590 of SEQ ID NO: 9), VFLAPWKK (SEQ ID NO: 19, amino acids 583 to 590 of SEQ ID NO: 9), FLAPWKK (SEQ ID NO: 20, amino acids 584 to 590 of SEQ ID NO: 9), LAPWKK (SEQ ID NO: 2, amino acids 585 to 590 of SEQ ID NO: 9), APWKK (SEQ ID NO: 21, amino acids 586 to 590 of SEQ ID NO: 9), PWKK (SEQ ID NO: 22, amino acids 587 to 590 of SEQ ID NO: 9), SKILTVRVILAPWKK (SEQ ID NO: 23, amino acids 587 to 601 of SEQ ID NO: 1), KILTVRVILAPWKK (SEQ ID NO: 24, amino acids 588 to 601 of SEQ ID NO: 1), ILTVRVILAPWKK (SEQ ID NO: 25, amino acids 589 to 601 of SEQ ID NO: 1), LTVRVILAPWKK (SEQ ID NO: 26, amino acids 590 to 601 of SEQ ID NO: 1), TVRVILAPWKK (SEQ ID NO: 27, amino acids 591 to 601 of SEQ ID NO: 1), VRVILAPWKK (SEQ ID NO: 4, amino acids 592 to 601 of SEQ ID NO: 1), RVILAPWKK (SEQ ID NO: 28, amino acids 593 to 601 of SEQ ID NO: 1), VILAPWKK (SEQ ID NO: 29, amino acids 594 to 601 of SEQ ID NO: 1), ILAPWKK (SEQ ID NO: 30, amino acids 595 to 601 of SEQ ID NO: 1), SSMRTSPRSSTLAVR (SEQ ID NO: 31, amino acids 568 to 582 of SEQ ID NO: 9), SSMRTSPRSSTLAV (SEQ ID NO: 32, amino acids 568 to 581 of SEQ ID NO: 9), SSMRTSPRSSTLA (SEQ ID NO: 33, amino acids 568 to 580 of SEQ ID NO: 9), SSMRTSPRSSTL (SEQ ID NO: 34, amino acids 568 to 579 of SEQ ID NO: 9), SSMRTSPRSST (SEQ ID NO: 35, amino acids 568 to 578 of SEQ ID NO: 9), SSMRTSPRSS (SEQ ID NO: 36, amino acids 568 to 577 of SEQ ID NO: 9), SSMRTSPRS (SEQ ID NO: 37, amino acids 568 to 576 of SEQ ID NO: 9), SSMRTSPR (SEQ ID NO: 38, amino acids 568 to 575 of SEQ ID NO: 9), SSMRTSP (SEQ ID NO: 39, amino acids 568 to 574 of SEQ ID NO: 9), SSMRTS (SEQ ID NO: 40, amino acids 568 to 573 of SEQ ID NO: 9), SSMRT (SEQ ID NO: 41, amino acids 568 to 572 of SEQ ID NO: 9), SSMR (SEQ ID NO: 42, amino acids 568 to 571 of SEQ ID NO: 9), NHASSVPRSKILTVR (SEQ ID NO: 43, amino acids 579 to 593 of SEQ ID NO: 1), NHASSVPRSKILTV (SEQ ID NO: 44, amino acids 579 to 592 of SEQ ID NO: 1), NHASSVPRSKILT (SEQ ID NO: 45, amino acids 579 to 591 of SEQ ID NO: 1), NHASSVPRSKIL (SEQ ID NO: 46, amino acids 579 to 590 of SEQ ID NO: 1), NHASSVPRSKI (SEQ ID NO: 47, amino acids 579 to 589 of SEQ ID NO: 1), NHASSVPRSK (SEQ ID NO: 48, amino acids 579 to 588 of SEQ ID NO: 1), NHASSVPRS (SEQ ID NO: 49, amino acids 579 to 587 of SEQ ID NO: 1), NHASSVPR (SEQ ID NO: 50, amino acids 579 to 586 of SEQ ID NO: 1), NHASSVP (SEQ ID NO: 51, amino acids 579 to 585 of SEQ ID NO: 1), NHASSV (SEQ ID NO: 52, amino acids 579 to 584 of SEQ ID NO: 1), NHASS (SEQ ID NO: 53, amino acids 579 to 583 of SEQ ID NO: 1), NHAS (SEQ ID NO: 54, amino acids 579 to 582 of SEQ ID NO: 1), TSPRSSTLAVRVFLA (SEQ ID NO: 55, amino acids 572 to 586 of SEQ ID NO: 9), SPRSSTLAVRVFL (SEQ ID NO: 56, amino acids 573 to 585 of SEQ ID NO: 9), PRSSTLAVRVF (SEQ ID NO: 57, amino acids 574 to 584 of SEQ ID NO: 9), RSSTLAVRV (SEQ ID NO: 58, amino acids 575 to 583 of SEQ ID NO: 9), SSTLAVR (SEQ ID NO: 59, amino acids 576 to 582 of SEQ ID NO: 9), STLAV (SEQ ID NO: 60, amino acids 577 to 581 of SEQ ID NO: 9), SVPRSKILTVRVILA (SEQ ID NO: 61, amino acids 583 to 597 of SEQ ID NO: 1), VPRSKILTVRVIL (SEQ ID NO: 62, amino acids 584 to 596 of SEQ ID NO: 1), PRSKILTVRVI (SEQ ID NO: 63, amino acids 585 to 595 of SEQ ID NO: 1), RSKILTVRV (SEQ ID NO: 64, amino acids 586 to 594 of SEQ ID NO: 1), SKILTVR (SEQ ID NO: 65, amino acids 587 to 593 of SEQ ID NO: 1), KILTV (SEQ ID NO: 66, amino acids 588 to 562 of SEQ ID NO: 1), RVFL (SEQ ID NO: 67, amino acids 582 to 585 of SEQ ID NO: 9), RVFLA (SEQ ID NO: 68, amino acids 582 to 586 of SEQ ID NO: 9), RVFLAP (SEQ ID NO: 69, amino acids 582 to 587 of SEQ ID NO: 9), RVFLAPW (SEQ ID NO: 70, amino acids 582 to 588 of SEQ ID NO: 9), RVFLAPWK (SEQ ID NO: 5, amino acids 582 to 589 of SEQ ID NO: 9), RVIL (SEQ ID NO: 71, amino acids 593 to 596 of SEQ ID NO: 1), RVILA (SEQ ID NO: 72, amino acids 593 to 597 of SEQ ID NO: 1), RVILAP (SEQ ID NO: 73, amino acids 593 to 598 of SEQ ID NO: 1), RVILAPW (SEQ ID NO: 74, amino acids 593 to 599 of SEQ ID NO: 1), RVILAPWK (SEQ ID NO: 7, amino acids 593 to 600 of SEQ ID NO: 1) RSKILTVRVILAPWK (SEQ ID NO: 75), SKILTVRVILAPWK (SEQ ID NO: 76), KILTVRVILAPWK (SEQ ID NO: 77), ILTVRVILAPWK (SEQ ID NO: 78), LTVRVILAPWK (SEQ ID NO: 79), and KGGGKWPALFVR (SEQ ID NO: 81). Said peptide may comprise at least one or at least one further chemical modification.

In one embodiment, the peptide consists of a sequence selected from the group consisting of: RVILAPWK (SEQ ID NO: 7), RVFLAPWK (SEQ ID NO: 5), and a retro-inverso sequence of one of these sequences. Said peptide may comprise at least one or at least one further chemical modification.

In another preferred embodiment of the invention, the initial peptide is selected in the group consisting of a peptide of sequence SEQ ID NO: 2, a peptide of sequence SEQ ID NO: 3, a peptide of sequence SEQ ID NO: 4, a peptide of sequence SEQ ID NO: 5, a peptide of sequence SEQ ID NO: 6 consisting of D-enantiomer amino acids, a peptide of sequence SEQ ID NO: 7 and a peptide of sequence SEQ ID NO: 8 consisting of D-enantiomer amino acids.

A more preferred initial peptide is a peptide of sequence SEQ ID NO: 5 (also called P8F) or a peptide of sequence SEQ ID NO: 6 consisting of D-enantiomer amino acids (also called P8RI).

The peptide may be prepared by any well-known procedure in the art, such as chemical synthesis, for example solid phase synthesis or liquid phase synthesis, or genetic engineering. As a solid phase synthesis, for example, the amino acid corresponding to the C-terminus of the peptide to be synthesized is bound to a support which is insoluble in organic solvents, and by alternate repetition of reactions, one wherein amino acids with their amino groups and side chain functional groups protected with appropriate protective groups are condensed one by one in order from the C-terminus to the N-terminus, and one where the amino acids bound to the resin or the protective group of the amino groups of the peptides are released, the peptide chain is thus extended in this manner. After synthesis of the desired peptide, it is subjected to the de-protection reaction and cut out from the solid support. Such peptide cutting reaction may be carried with hydrogen fluoride or tri-fluoromethane sulfonic acid for the Boc method, and with TFA for the Fmoc method.

Solid phase synthesis methods are largely classified by the tBoc method and the Fmoc method, depending on the type of protective group used. Typically used protective groups include tBoc (t-butoxycarbonyl), CI—Z (2-chlorobenzyloxycarbonyl), Br—Z (2-bromobenzyloyycarbonyl), BzI (benzyl), Fmoc (9-fluorenylmcthoxycarbonyl), Mbh (4,4′-dimethoxydibenzhydryl), Mtr (4-methoxy-2,3,6-trimethylbenzene-sulphonyl), Trt (trityl), Tos (tosyl), Z (benzyloxycarbonyl) and Clz-BzI (2,6-dichlorobenzyl) for the amino groups; NO2 (nitro) and Pmc (2,2,5,7,8-pentamethylchromane-6-sulphonyl) for the guanidino groups; and tBu (t-butyl) for the hydroxyl groups.

Alternatively, the CD31 peptide may be synthesized using recombinant techniques.

The method of producing the peptide may optionally comprise the steps of purifying said CD31-derived peptide, chemically modifying said CD31-derived peptide, and/or formulating said CD31-derived peptide into a pharmaceutical composition.

According to a preferred embodiment, the CD31-derived peptide comprising an azide terminal group comprises a peptide of sequence KWPALFVR (SEQ ID NO: 6), wherein the amino acids are D-enantiomer amino acids.

Preferably, the initial peptide consists of sequence KWPALFVR (SEQ ID NO: 6), wherein the amino-acids are D-enantiomer amino acids, and the CD31-derived peptide comprising an azide terminal group thus comprises the sequence KWPALFVR (SEQ ID NO: 6) consisting of D-enantiomer amino acids and an azide terminal group.

A preferred CD31-derived peptide comprising an azide terminal group according to the invention consists of:
(i) a spacer at the N-terminus end, for example a spacer of sequence KGGG (SEQ ID NO: 80) consisting of D-enantiomers amino acids, and
(ii) the sequence KWPALFVR (SEQ ID NO: 6) consisting of D-enantiomers amino acids at the C-terminal end,
the N-terminus of the spacer being an azide group (i.e. presence of an azide group —N═N⁺═N⁻ instead of the amino group).

According to a preferred embodiment, the CD31-derived peptide comprising an azide terminal group comprises a peptide having the sequence KGGGKWPALFVR (SEQ ID NO: 81), wherein the amino-acids are D-enantiomers amino acids.

Preferably, the CD31-derived peptide comprising an azide terminal group has the sequence KGGGKWPALFVR (SEQ ID NO: 81) with an azide terminal group, said peptide consisting of D-enantiomer amino acids. A preferred CD31-derived peptide comprising an azide terminal group according to the invention has the sequence KGGGKWPALFVR (SEQ ID NO: 81), the N-terminus of which being an azide group (presence of an azide group —N═N⁺═N⁻ instead of the amino group) and said peptide consisting of D-enantiomer amino acids.

For example, the CD31-derived peptide comprising an azide terminal group has the following formula:

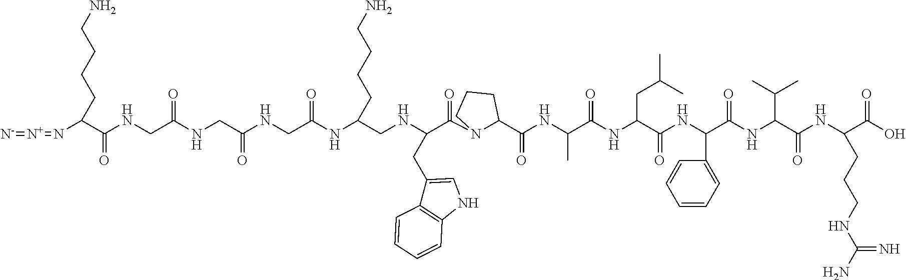 [SMILES: CC(C)CC(NC(=O)C(C)NC(=O)C1CCCN1C(=O)C(Cc1c[nH]c2ccccc12)NCC(CCCCN)NC(=O)CNC(=O)CNC(=O)CNC(=O)C(CCCCN)N=[N+]=[N-])C(=O)NC(C(=O)NC(C(=O)NC(CCCNC(=N)N)C(=O)O)C(C)C)c1ccccc1]

According to an embodiment, the surface (or platform) of the device as mentioned above is made of metals or metal alloys, preferably a stainless steel, cobalt-chromium (CoCr) alloy, platinum-chromium (PtCr) alloy or a nickel-titanium alloy (such as Nitinol).

According to the invention, the device is a metallic implantable device for interventional neuroradiology.

Preferably, the device for interventional neuroradiology is chosen from the group consisting of: intracranial stents, flow-diverter stents, and metallic embolization devices.

According to a preferred embodiment, the present invention concerns a method for the modification of a device surface by grafting a CD31-derived peptide onto the surface of said device, said CD31-derived peptide and device being as defined above, wherein the device is a stent, preferably a flow diverting stent, made of metals or metal alloys, in particular made of a nickel-titanium alloy (such as Nitinol).

The present invention also relates to a modified surface device as defined above, wherein the surface of said device is grafted by a CD31-derived peptide as defined above, obtainable by the method as mentioned above.

Preferably, this device has a modified surface comprising a coating made of a layer of polydopamine and a layer made of the linker and the CD31-derived peptide as defined above.

### BRIEF DESCRIPTION OF THE SEQUENCES

SEQ ID NO: 1 corresponds to the sequence of human CD31.

SEQ ID NO: 2 corresponds to the sequence LAPWKK of a 6 amino acid peptide derived from human or murine CD31.

SEQ ID NO: 3 corresponds to the sequence VRVFLAPWKK of a 10 amino acid peptide derived from murine CD31, also called PepReg CD31.

SEQ ID NO: 4 corresponds to the sequence VRVILAPWKK of a 10 amino acid peptide derived from human CD31.

SEQ ID NO: 5 corresponds to the sequence RVFLAPWK of a 8 amino acid peptide derived from murine CD31, also called P8F.

SEQ ID NO: 6 corresponds to the sequence KWPALFVR of 8 amino acids and also correspond to the inverted sequence of SEQ ID NO: 5. When a peptide of sequence SEQ ID NO: 6 consists of D-amino acids, this peptide kwpalfvr is also called P8RI.

SEQ ID NO: 7 corresponds to the sequence RVILAPWK of a 8 amino acid peptide derived from human CD31.

SEQ ID NO: 8 corresponds to the inverted sequence of SEQ ID NO: 7.

SEQ ID NO: 9 corresponds to the sequence of murine CD31.

SEQ ID NO: 10 corresponds to the sequence of bovine CD31.

SEQ ID NO: 11 corresponds to the sequence of pig CD31.

SEQ ID NO: 12 corresponds to the amino acids 579 to 601 of sequence SEQ ID NO: 1.

SEQ ID NO: 13 to 79 correspond to CD31-derived sequences.

SEQ ID NO: 80 corresponds to sequence of the spacer KGGG.

SEQ ID NO: 81 corresponds to the sequence of a peptide comprising the spacer of sequence SEQ ID NO: 80 and the CD31-derived peptide of sequence SEQ ID NO: 6.

### BRIEF DESCRIPTION OF THE DRAWINGS

FIG. 1. Elemental composition of PDA-linker-P8RI coated samples after ageing in PBS. A. Surface atomic percentages. B. N/C ratio of the samples. Error bars: standard deviation over 3 replicates.

FIG. 2. Hemolysis ratios after 1 h (left) or 24 h incubation (right). No significant difference appear between the negative control and the bare and coated disks, after 1 h or 24 h of incubation.

FIG. 3. Results of a multiplex assay analysis of cell culture supernatants from a representative experiment performed with HUVECs. Absolute concentrations (in pg/mL) are expressed. *: p<0.05. **: p<0.01. ***: p<0.001.

FIG. 4. Results of multiplex assay analyses of cell culture supernatants. Each point is a biological replicate. For each analyte and each experiment, concentrations were normalized with respect to “CoCr” group.

FIG. 5. SEM images of a FDS implanted in the subclavian artery of a rabbit after each step of the coating procedure (Bare: before the coating; PDA: after step a); PDA+BCN: after step b); and PDA+BCN+P8RI: after step c)). The final coating is thin smooth and evenly distributed over the stent struts.

FIG. 6. Fluorescence microscopy images of FDS struts (black) incubated with whole human peripheral blood for one hour at 37° C. CD41 staining (white) reveals the platelets on the stent struts. Diffuse and partially aggregated platelets adhere to the controls whereas this process seems limited on P8RI coated FDS.

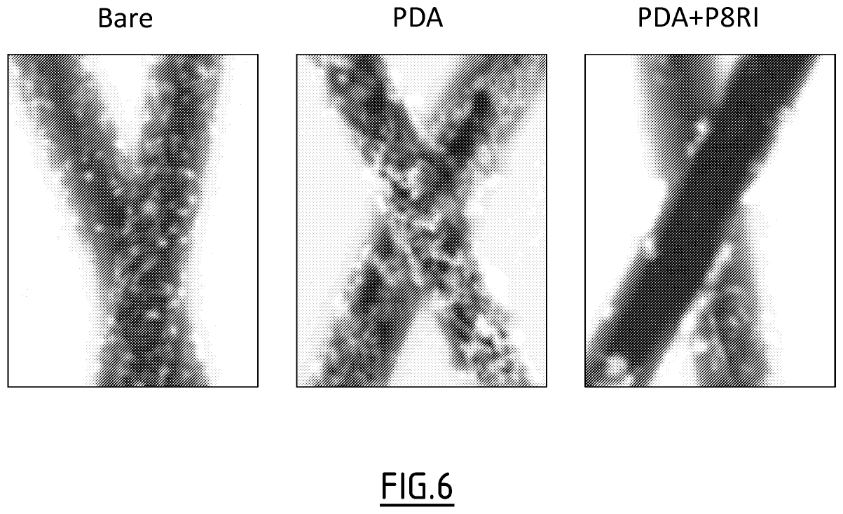

FIG. 7. SEM images of FDS implanted in the subclavian artery of a rabbit (magnification: 25×, 250×, and 1000×). The luminal side of the half-artery is shown. One month after implantation, the FDS are entirely covered with arterial tissue. The tissue covering PDA+P8RI coated FDS appeared smoother as compared to the control.

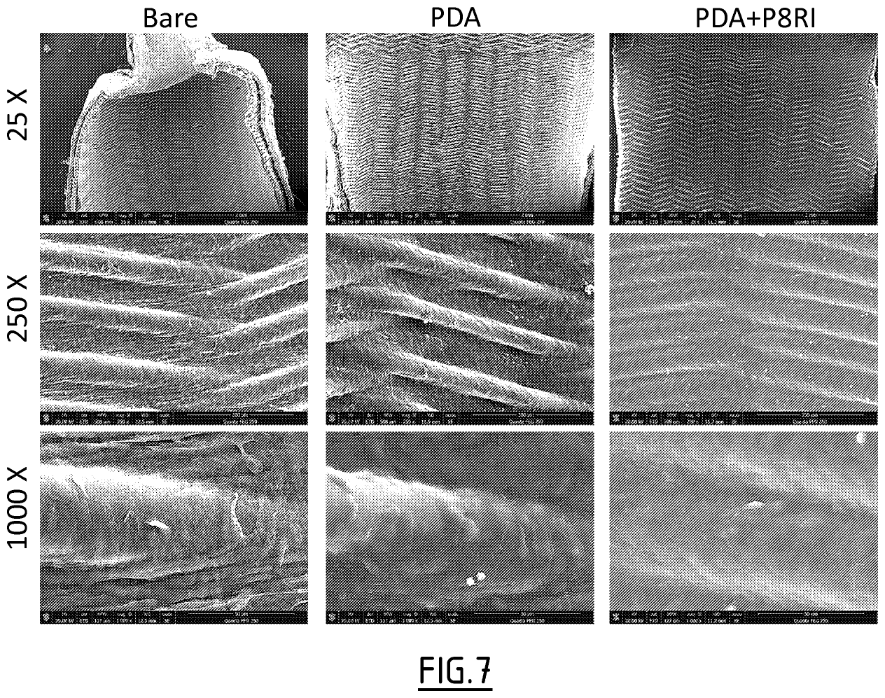

FIG. 8. Trichrome staining of stented rabbit experimental aneurysm (resin cross sections). The asterisks indicate the location of the stent struts. Light grey=Connective tissue, dark gray=cytoplasm, black=nuclei. “An” indicates the aneurysmal sac. Details of the neointima formed in front of the aneurysmal sac (top insets) show that the arterial tissue covering the stent struts is better organized in the P8RI FDS as documented by the presence of a continuous layer of endothelial cells covering the underlying smooth stromal cells which are oriented in the sense of the cyclic arterial stretch.

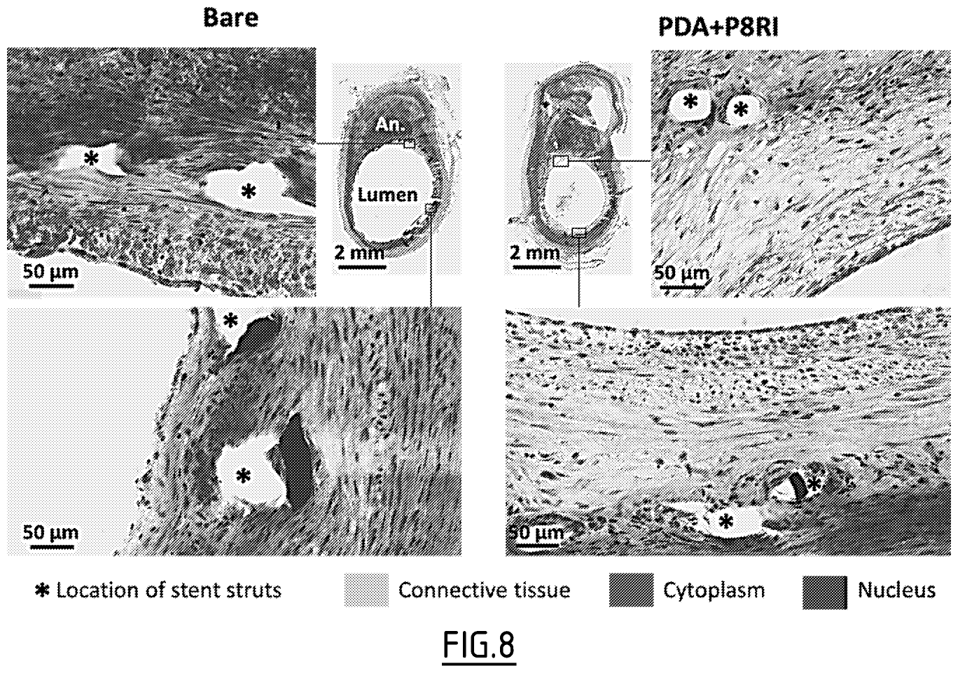

### DESCRIPTION OF THE PREFERRED EMBODIMENTS

Preparation of Modified Devices

1. Deposition of a Polydopamine Layer

The first step was the deposition of a self-assembled polydopamine layer on the L-605 CoCr samples.

The CoCr disks were made from mirror-polished L-605 sheets processed by the manufacturer (Goodfellow Cambridge Ltd). Thus, these CoCr disks were free of zinc contaminations, and presented a very flat surface.

The CoCr disks were ultrasound cleaned in three successive 10-minute baths of acetone, ethanol and deionized water. This cleaning procedure ensured the removal of organic contaminants. They were then put in an acid bath (40% HNO₃) for 40 minutes, in order to remove any ionic deposits from the alloy surface and to passivate it. After extensive washing with deionized water, the disks were sterilized by a 10-minute incubation in 70% ethanol. All subsequent steps were performed in sterile conditions, in a laminar flow hood. Solutions were filtered before use. The samples were rinsed several times with water, and, for the last wash, with the Tris buffer used for the dopamine solution.

The following coating protocol was adapted from the work by Lee and colleagues (Lee, H., Dellatore, S. M., Miller, W. M. and Messersmith, P. B. 2007. Science (New York, N.Y.) 318 (5849): 426-430).

A 2 mg/mL dopamine solution was prepared just before use from lyophilized dopamine hydrochloride (Alfa Aesar A11136) dissolved in a 10 mM Tris-HCl aqueous buffer with a pH adjusted to 8.5. The solution was added to the wells of a sterile multi-well plate containing the individual experimental samples. The plate containing the samples and the dopamine solution were thereafter covered with a sterile lid and incubated at room temperature for 22±2 h, under orbital shaking and protected from light. This incubation time resulted in a uniform coating, approximately 45 nm thick. The samples were then thoroughly rinsed with deionized water, placed in an ultrasound bath for 5 minutes in order to remove polydopamine aggregates from the surface, and further rinsed with deionized water.

The chemical composition of the PDA (polydopamine) coating was analyzed by Fourier Transform Infrared (FTIR) spectroscopy.

Quantitative information on the elemental composition of the PDA coating was obtained by XPS. XPS spectra were recorded on bare and PDA-coated CoCr disks, using a PHI 5600-CI spectrometer (Physical Electronics). Survey spectra were acquired with a monochromatic aluminum X-ray source (300 W) whereas high-resolution spectra were recorded with a monochromatic magnesium X-ray source (300 W). The detection angle was set to 45°. The analysis was done on three spots per disk to assess the homogeneity of the coating. We analyzed the spectra with the software MultiPak (Physical Electronics).

The dopamine solution is originally clear and turns light pink and finally black, as the polymerization process takes place and polydopamine aggregates form. PDA-coated CoCr disks also exhibit a dark brown color.

In the FTIR spectra, several peaks and one band were found on the PDA spectrum and not on the bare CoCr spectrum, all attributed to vibrations of chemical bonds that are present in polydopamine. Those attributions are in agreement with the literature (Xi, Z.-Y., Xu, Y.-Y., Zhu, L.-P., Wang, Y. and Zhu, B.-K. 2009. Journal of Membrane Science 327 (1): 244-253; Yu, F., Chen, S., Chen, Y., Li, H., Yang, L., Chen, Y. and Yin, Y. 2010. Journal of Molecular Structure 982(1): 152-161; Zhou, Y., Weng, Y., Zhang, L., Jing, F., Huang, N. and Chen, J. 2011. Applied Surface Science 258 (5): 1776-1783; Luo, R., Tang, L., Wang, J., Zhao, Y., Tu, Q., Weng, Y., Shen, R. and Huang, N. 2013. Colloids and Surfaces B-Biointerfaces 106: 66-73) and evidence the successful deposition of a polydopamine coating on the disks.

The below table shows the average surface atomic percentages of bare and PDS-coated CoCr disks (standard deviation between three replicates):

| | O 1 s | C 1 s | N 1 s | Cr 2p3 | Co 2p3 | W 4f | Ni 2p3 |
|---|---|---|---|---|---|---|---|
| CoCr | 44.3 ± 1.1 | 41.9 ± 0.8 | | 10.4 ± 0.5 | 1.8 ± 0.2 | 1.4 ± 0.1 | 0.3 ± 0.3 |
| PDA | 21.1 ± 0.3 | 71.1 ± 0.7 | 7.8 ± 0.5 | | | | |

On the bare samples (CoCr), the main elements that compose the L-605 alloy were detected (chromium, cobalt, tungsten and nickel). The presence of oxygen and carbon can be explained by the presence of metal oxides and (to a lesser extent) carbides, as well as residual surface organic contaminants.

On the PDA-coated disks (PDA), no metallic element was detected. That indicates that the PDA coating covered the surface in a uniform and complete manner. The only elements that were detected were oxygen, carbon and nitrogen, which compose the dopamine molecule. They were found in proportions close to those of the dopamine molecule (18% oxygen, 73% carbon, 9% nitrogen). These results therefore confirm the results obtained with the FTIR characterization.

3. Preparation of the Peptide P8RI

A CD31-derived peptide having an 8-residue long sequence, included in the original 23-residue long peptide, was chosen because it is highly conserved among species and because it showed high specificity towards CD31 in in vitro assays. The CD31 peptide was designed with D-amino acids. Thus, the peptidic sequence was assembled in reverse order so as to retain the original spatial orientation and the chirality of the side chains. The resulting peptide was named P8RI′, where P stands for peptide, 8 is the number of amino acids, and RI means retro-inverso. The sequence of P8RI is:

(D-Lys)-(D-Trp)-(D-Pro)-(D-Ala)-(D-Leu)-(D-Phe)-(D-Val)-(D-Arg).

The structure of P8RI is shown below, along with that of the original, non-inverted sequence (called P8F).

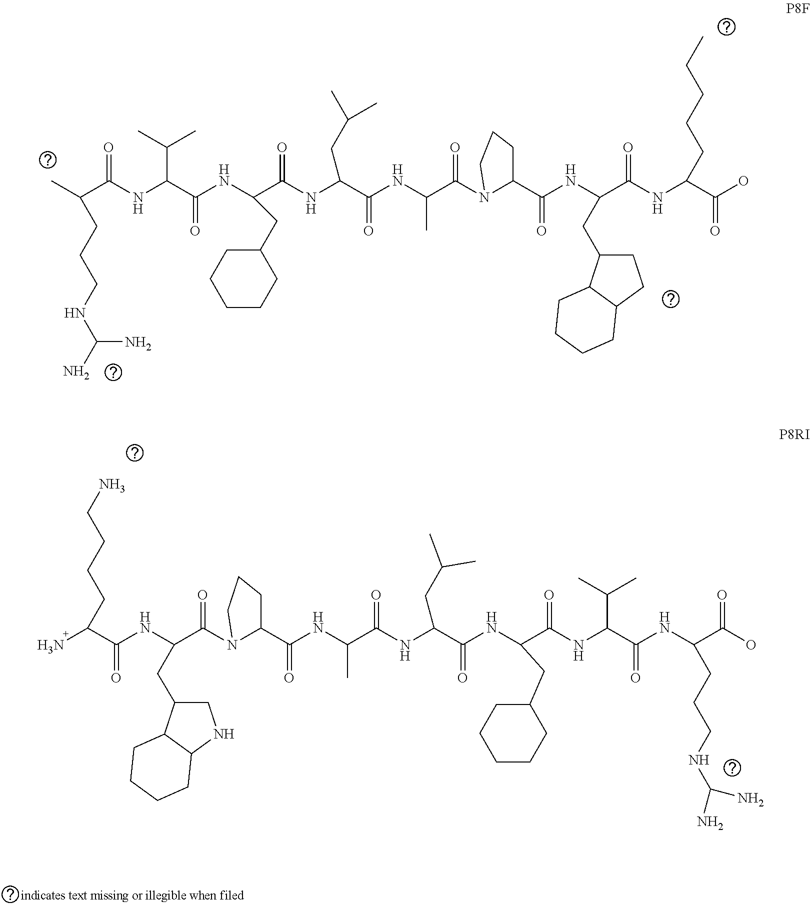 [SMILES: CCCCCC(NC(=O)C(CC1CCC2CCCCC21)NC(=O)C1CCCN1C(=O)C(C)NC(=O)C(CC(C)C)NC(=O)C(CC1CCCCC1)NC(=O)C(NC(=O)C(C)CCCNC(N)N)C(C)C)C(=O)O ; CC(C)CC(NC(=O)C(C)NC(=O)C1CCCN1C(=O)C(CC1CNC2CCCCC12)NC(=O)C(N)CCCCN)C(=O)NC(CC1CCCCC1)C(=O)NC(C(=O)NC(CCCNC(N)N)C(=O)O)C(C)C]

‘P8F’ designates the original sequence from the CD31 molecule, in forward sense, whereas P8RI is the retro-inverso sequence, where both the order and the chirality of the residues are inversed.

4. Immobilization of the Peptide

In order to better control the orientation of the immobilized P8RI and to render it readily accessible to its ligands at the surface of the cells contacting the coated surface, the inventors used a flexible linker as an intermediary between the peptide and the PDA coating.

The linker needs to have: 1) either an amine or a thiol function at one extremity, to be able to react with the o-quinone functions of the polydopamine coating, 2) a flexible chain to improve the accessibility of the bound P8RI and 3) a function at the second extremity that would specifically bind to the peptide N-terminus. As regards that last point, the inventors opted for a bioorthogonal reaction so as to avoid any interference from the side chains of the peptide and the amine functions of the polydopamine.

Copper-free click chemistry was used as it allows for a fast reaction in aqueous solution, without the addition of cytotoxic catalysts such as copper.

The strain-promoted azide-alkyne cycloaddition (SPAAC) has been carried out:

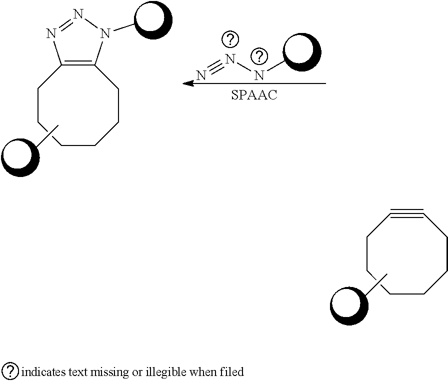

This addition has been carried out with an azide on the peptide and a bicyclo[6.1.0]nonyne (BCN) on the linker.

A “P8RI azide” was custom-synthesized, using a modified lysine with an azide instead of the side chain amine was introduced at the N-terminus of the peptide, and separated from the P8RI sequence by three glycines. The sequence of the “P8RI azide” was therefore:

Lys(N₃)-Gly-Gly-Gly-(D-Lys)-(D-Trp)-(D-Pro)-(D-Ala)-(D-Leu)-(D-Phe)-(D-Val)-(D-Arg).

Concerning the linker, we opted for a short PEG chain between the BCN at one end and an amine at the other end. PEG was chosen for its high flexibility and biocompatibility. The resulting linker (Sigma 745073) will be called “BCN-amine” throughout this thesis.

The two-step immobilization of the P8RI azide on the PDA coating through the BCN-amine linker is as follows:

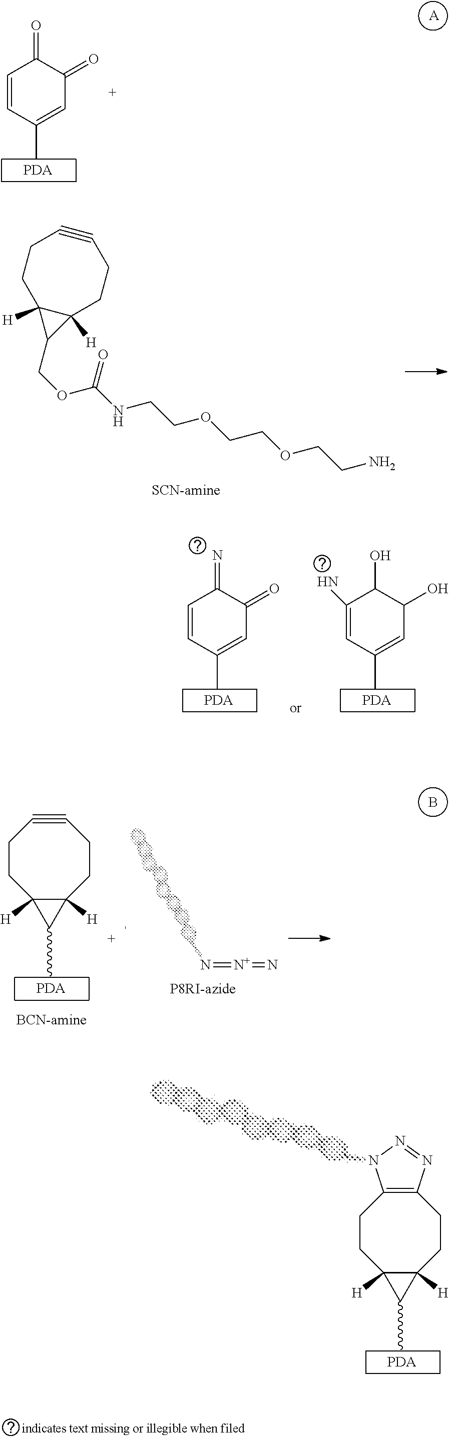

A. Binding of the BCN-amine linker to the PDA coating by Schiff base reaction (left) or Michael addition (right).

B. Binding of the peptide to the immobilized BCN-amine linker by click chemistry.

5. Immobilization of P8RI Azide on PDA Coatings with the Help of the BCN-Amine Linker

BCN-amine binding was carried out on CoCr disks immediately after the polydopamine coating procedure, in a laminar flow hood. A solution containing 0.1 mg/mL BCN-amine (Sigma 745073) diluted in Tris buffer (10 mM Tris, pH adjusted to 8.5) was added to the wells containing the PDA-coated samples. They were incubated for 22±2 h under orbital shaking. After thorough rinsing with deionized water, a solution containing 0.2 mg/mL “P8RI azide” in deionized water was added to the samples. The samples were thenthoroughly rinsed with deionized water.

Fluorescence microscopy and XPS analysis of untagged peptide immobilized on the surface were performed.

For the XPS analysis, unmodified “P8RI azide” was immobilized on CoCr disks as described above. The coated samples were then analyzed by XPS as detailed above. Three points were analyzed on each sample.

For the fluorescence microscopy analysis, a “P8RI azide” with an additional C-terminal lysine conjugated to a FITC fluorophore was custom synthesized by the manufacturer Proteogenix. The pseudo-peptidic sequence of that “P8RI-FITC azide” thus was:

Lys(N₃)-Gly-Gly-Gly-(D-Lys)-(D-Trp)-(D-Pro)-(D-Ala)-(D-Leu)-(D-Phe)-(D-Val)-(D-Arg)-Lys(FITC).

The “P8RI-FITC azide” was immobilized on a BCN-amine linker on PDA-coated CoCr disks as described for the “P8RI azide”. The coated samples were then placed face down in imaging dishes to prepare their observation through an inverted microscope and covered with mounting medium (ProLong Gold Antifade Mountant, Thermo Fisher P36930). This medium has a refractive index close to that of the material through which the samples are observed (glass or plastic), so that light transmission is optimized. The ProLong Gold mounting medium also has antifading properties that minimize photobleaching, thus allowing the preservation of fluorescent samples for longer times. Digital photographs of the samples were acquired on an Axio Observer inverted fluorescence microscope (Zeiss), equipped with the software Zen (Zeiss).

CoCr disks on which the “P8RI-FITC azide” had been immobilized (following the protocol described above) emitted a green fluorescence of much higher intensity than the PDA-coated disks. These results prove the presence of the fluorescent peptide at the surface of the disks after the immobilization protocol.

The XPS analysis of the surface of coated CoCr disks revealed only the presence of the three elements nitrogen, oxygen and carbon on the three types of coating: PDA only, PDA+BCN-amine linker, PDA+linker+P8RI azide. This was consistent with the previous analyses of PDA coatings, which had appeared to be homogeneous. Atomic ratios of nitrogen and oxygen over carbon were calculated from the XPS spectra. They are presented in the below table, along with the theoretical values of these ratios for the molecules involved in the coatings:

| | | O/C | N/C |
|---|---|---|---|
| Values measured by XPS | PDA | 0.297 ± 0.007 | 0.111 ± 0.008 |
| on coated CoCr disks | PDA-linker | 0.282 ± 0.003 | 0.106 ± 0.011 |
| | PDA-linker- | 0.254 ± 0.005 | 0.120 ± 0.007 |
| | P8RI | | |
| Values calculated from | Dopamine | 0.250 | 0.125 |
| the molecular formulas | Linker | 0.235 | 0.118 |
| | P8RI azide | 0.211 | 0.316 |

The O/C ratio of the polydopamine samples is slightly higher, and the N/C ratio slightly lower, than the values of the pure dopamine molecule. The evolution of the ratios with the addition of the linker and the P8RI to the coatings follows the tendency of the theoretical values: a constant decrease of the O/C ratio, and a slight decrease followed by a larger increase of the N/C ratio. Therefore, these results point to the successful immobilization of the BCN-amine linker and of the P8RI azide.

Stability of the PDA-Linker-P8RI Coating in One-Month Ageing Test in Liquid Medium

In order to have its full effect in vivo, the “PDA-linker-P8RI” coating needs to maintain its integrity for the time necessary for stent endothelialization (about one week). The ageing behavior of the coating was assessed by an in vitro static ageing test in liquid medium. PBS was chosen as the liquid medium. Each CoCr disk was fitted in a custom sample holder designed to expose only its coated surface to PBS. The floating sample holder was placed in a beaker filled with PBS and stored in an incubator at 37° C. (human core body temperature) for 1 or 4 weeks. Before the test, the beakers and sample holders were sterilized by autoclave to prevent bacterial proliferation during the study. The samples were manipulated in sterile conditions, under a laminar flow hood. After ageing, the disks were removed from their holders, thoroughly rinsed with deionized water, and dried with medical-grade compressed air. They were then analyzed by XPS. Three coated disks and one bare CoCr disk were tested for each time point (1 or 4 weeks).

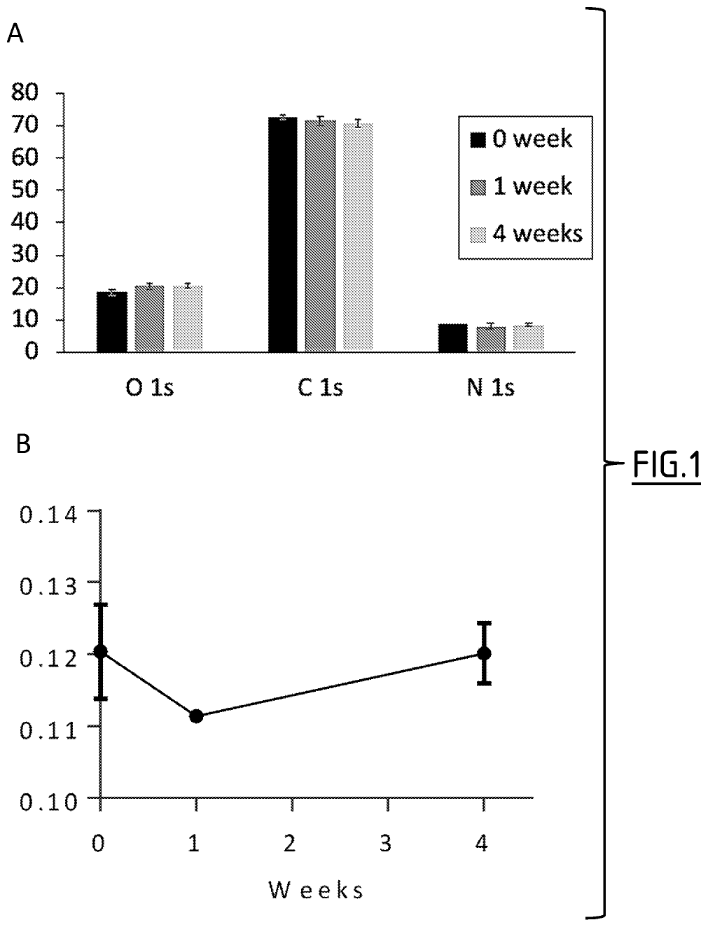

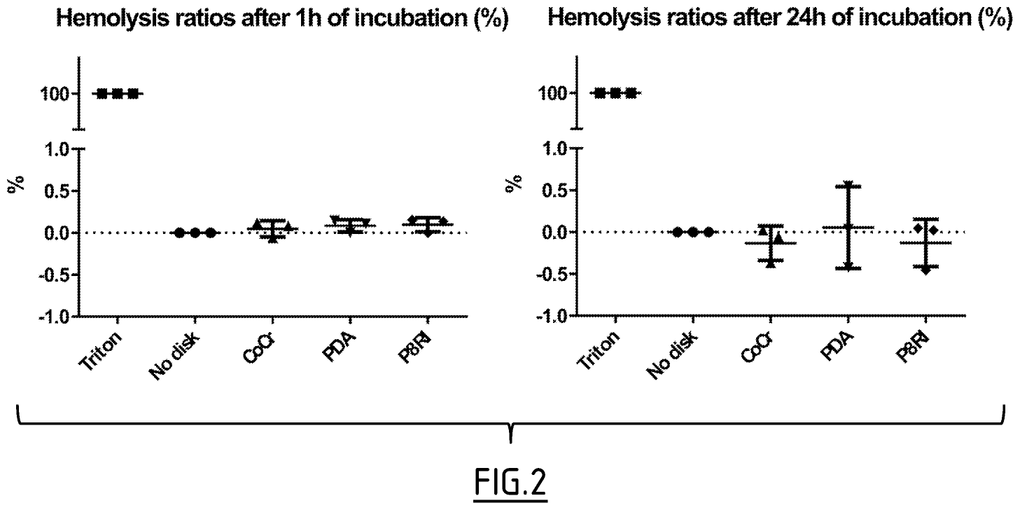

The surface atomic percentages of the coatings measured by XPS are presented on FIG. 1A. The N/C ratio was considered as the most reliable indicator of the potential degradation of the coating since it is not influenced by oxidation or water adsorption on the coating. As shown on FIG. 2B, the N/C ratio of the “PDA-linker-P8RI” coating was unmodified after 4 weeks of ageing in PBS at 37° C., thus demonstrating that the coating did not undergo any significant degradation.

In Vitro Evaluation of the Biocompatibility of P8RI-Coated Surfaces

In the subsequent paragraphs, ‘CoCr’ designates bare L-605 CoCr disks, ‘PDA’ means polydopamine-coated CoCr disks, and P8RI′ are CoCr disks with a polydopamine-linker-P8RI coating.

The concentration of P8RI used during the last step of the polydopamine-linker-P8RI coating process was chosen on the basis of a dose-effect curve obtained using 4-fold dilutions between 6 and 200 μg/mL. In preliminary experiments, the concentration of 50 μg/mL yielded the least pro-inflammatory (as detected by the production of soluble IL-6 and VCAM-1) and the most anti-thrombotic (based on the levels of soluble TFPI) phenotype of primary human endothelial cells cultured on the coated surfaces The concentration of 50 μg/mL was therefore used for coating the CoCr samples in the in vitro experiments presented in the following paragraphs, and the stents in the preclinical studies as explained hereafter.

1. Hemocompatibility

1.1. No Hemolysis

The hemolysis assay is a required biocompatibility test for all blood-contacting devices. It shows whether a given material causes erythrocyte lysis (either by contact or by the release of toxic molecules). The hemolysis assay protocol we used was adapted from the one used by Bae and coworkers (Bae, I.-H., Park, I.-K., Park, D. S., Lee, H. and Jeong, M. H. 2012. Journal of Materials Science: Materials in Medicine 23 (5): 1259-1269.) as detailed below.

Coated and bare CoCr disks were placed in the wells of a 96-well plate and sterilized by a ten minute incubation in 70° ethanol. The disks were rinsed several times in water prior to a final wash in physiological saline solution (0.9% NaCl). Human peripheral whole blood collected in lithium heparin (18 Ul/mL) was then gently layered on each disk and the plate was incubated at 37° C. during either 1 h or 24 h. Heparin was chosen for anticoagulation because it does not interfere with the hemolysis assays.

Disk-free wells were used as negative controls and 1% Triton X-100 (which causes the lysis of most erythrocytes through the dissolution of their plasma cell membrane) was added in blood-containing positive control wells. At the end of the incubation period, the blood was transferred from each well to an individual polypropylene tube (Eppendorf, 1.5 ml) and centrifuged at 1200 g for 15 minutes. 50 μL of platelet-poor plasma were collected at the top of all centrifuged tubes and transferred to a new 96-well plate. Absorbance was measured at 540 nm in a plate reader spectrophotometer (Infinite 200 PRO, TECAN). 540 nm corresponds to the absorbance peak of free hemoglobin, which is released by hemolysis from the erythrocytes. The value of the absorbance at 540 nm is therefore directly proportional to the extent of hemolysis caused by the sample disks in the experimental wells.

The experiment was performed in technical quadruplicates and repeated three times using the blood of different healthy blood donors.

According to the photograph of the microplate taken just before the measurement of the absorbance at the end of a hemolysis assay, the platelet-poor plasma from whole blood incubated with Triton X-100 appears dark red, reflecting the effective hemolysis in these wells (positive controls). No macroscopic difference in color is apparent between wells containing the negative control (No disk) and the three experimental groups of plasma.

For each experiment, these observations were quantified by absorbance reading at 540 nm, and the measurements were normalized using the values obtained with the positive and negative controls, according to the following formula:

$$R ( x ) = \frac{{A}_{540 nm} ( x ) - {A}_{540 nm} ( No disk )}{{A}_{540 nm} ( Triton ) - {A}_{540 nm} ( No disk )}$$

wherein R(x) is the normalized ratio of group x, and A_{540 nm}(x) is the average absorbance of the wells from group x. The measured ratios are presented on FIG. 2.

In the three experimental groups (blood incubated with the CoCr disks), after 1 h or 24 h or incubation, the hemolysis ratios were not significantly different from the negative controls.

Therefore, it can be concluded that neither the bare CoCr surface nor the coated CoCr surfaces that were used were evidenced to cause hemolysis when contacted with human whole blood.

1.2. Platelet Adhesion

As thrombosis is one of the two main complications associated with stenting, evaluating the thrombogenicity of each novel stenting material is essential. To this aim, we performed a platelet adhesion assay.

Coated and bare CoCr disks were incubated in 70° ethanol and rinsed, as detailed above. Human whole blood collected in PPack (75 mM PPACK+0.1% D-mannitol, Haemtech SCAT-875B) was then deposited on the disks and they were incubated at 37° C. for 1 h. PPack (Phe-Pro-Arg-chloromethylketone) is a peptidomimetic thrombin inhibitor that inhibits the coagulation cascade without affecting the physiological concentration of ionized calcium. This is important in a functional test such as platelet adhesion, which is a Ca++ dependent process. At the end of the incubation period, the disks were taken out of the wells using delicate tweezers and rinsed by gently stirring in a beaker full of physiological saline solution at room temperature. They were then placed in a new 96-well microplate and rinsed twice in physiological saline solution, prior to fixation with paraformaldehyde. The disks were then processed for immunocytofluorescence.

In order to visualize all adherent platelets, we chose to immunostain two antigens that are constitutively expressed by platelets: CD41 and von Willebrand Factor (vWF). CD41, also known as Integrin alpha-IIb, is the most abundant platelet adhesion receptor and is thus present on the surface of the platelets. vWF is a glycoprotein which plays a major role in blood coagulation. As it is stored in intracellular compartments of platelets (the α-granules), its immunostaining requires the permeabilization of the platelet membrane. Since this “platelet adhesion assay” was performed with whole blood, leukocyte adhesion was also possible. Hoechst staining of cell nuclei was used to identify leukocytes (which, contrary to platelets, possess a cell nucleus).

The reagents used in the following protocol are presented on the below table.

| Designation | Reference | Concentration | |
|---|---|---|---|
| Bovine serum albumin | Jackson | 5% | m/v |
| | ImmunoResearch | | |
| | 001-000-173 | | |
| Fish gelatin | Sigma-Aldrich | 0.1% | m/v |
| | F7041 | | |
| Mouse anti-human CD41 | Beckman-Coulter | 2 | μg/mL |
| primary antibody | IM045 | | |
| Rabbit anti-human vWF | Dsko | 30 | μg/ml |
| primary antibody | A0082 | | |
| Goat anti-mouse IgG, | Jackson | 3.75 | μg/mL |
| Cyanine3 secondary | ImmunoResearch | | |
| antibody | 115-166-871 | | |
| Goat anti-rabbit IgG, | Jackson | 3.75 | μg/mL |
| Alexa Fluor 488 secondary | ImmunoResearch | | |
| antibody | 111-546-046 | | |
| Hoechst 33342 | Invitrogen | 10 | μg/mL |
| | H3570 | | |
| Prolong Gold Antifade | Thermo Fisher | | |
| Mountant | P36930 | | |

The experimental samples were fixed in 4% paraformaldehyde at 4° C. for 10 minutes, then rinsed 3 times in Dulbecco's phosphate buffered saline (PBS). Fixation protects biological samples from decay by cross-linking the proteins. The samples were then permeabilized by a 10-minute incubation in a solution containing 100 mM glycine and 0.5% Triton X-100 in PBS. Triton X-100, being a non-ionic detergent, creates pores in the cell membranes without denaturing proteins, while glycine was used to quench the formaldehyde. After PBS rinsing, blocking was then performed by a 30-minute incubation in a solution of 5% bovine serum albumin and 0.1% fish gelatin in PBS, in order to reduce unspecific antibody binding, and thus decrease background noise.

The two primary antibodies were diluted in a solution containing 1% bovine serum albumin (BSA) and 0.02% fish gelatin in PBS, and incubated overnight at 4° C. with the samples. The samples were then rinsed with PBS and the secondary antibodies, diluted in a similar fashion, were added and incubated for 1 hour at room temperature. After PBS rinsing, the nuclei of the cells were stained by incubation in Hoechst solution. Finally, the samples were placed face down in imaging dishes (to prepare their observation by inverted microscope) and covered with Prolong Gold mounting medium. Digital photographs of the immunostained samples were then acquired on an Axio Observer inverted fluorescence microscope (Zeiss), equipped with the software Zen (Zeiss). The surface of the disks covered by platelets was identified by positive CD41 and vWF staining and quantified on the digital images using the “Analyze particles” function of the open source software Fiji.

The observation of the resulting photographs showed that very few leukocytes had adhered to the surface of the samples (where they were easily identified by their Hoechst positive nuclei). Globally, the surface density of platelets (identified by positive CD41 and vWF staining) appeared lower on PDA than on bare CoCr, and even lower on “P8RI” disks as compared to the “PDA” ones. However, high variability can be noted between different disks of the same group, and this was reflected by the values obtained by computer-assisted quantification. Therefore, the significance of the differences observed between the experimental groups is questionable. The experiment was repeated several times, but, as intra-group variability was always high, no significant difference in the adhered platelets density was found between the three groups.

It can be concluded from these experiments that the “PDA” and “P8RI” coatings do not increase but rather tend to reduce platelet adhesion on the surface of CoCr samples.

2. Endothelialization

A functional evaluation of coronary artery endothelial cells upon their contact with the experimental (bare and coated) CoCr disks described before was performed. Attachment and survival were evaluated by computer-assisted analysis of digital images after immunofluorescent staining whereas the pro-inflammatory and pro-thrombotic phenotypes were assessed through quantitative analysis of soluble biomarkers in cell culture supernatants.

For practical reasons, the first experiments were performed with human umbilical vein endothelial cells (HUVECs, from PromoCell). Human coronary artery endothelial cells from three different individual donors (HCAECs, purchased from Lonza) were then used, in order to better reproduce the environment of coronary stents. The cells were cultured in Endothelial cells Growth Medium MV2 (Promocell), which contained the nutrients and growth factors needed by ECs. Antibiotic, antifungal and antimycoplasma reagents (penicillin, streptomycin, amphotericin B, plasmocin and primocine) were added to the medium in order to prevent contaminations. The cells were used at passages 3 to 5.

Coated and uncoated CoCr disks were placed in the wells of a 96-well plate and sterilized as described above. 100000 endothelial cells suspended in their growth medium were seeded on each disk. After a 48 h incubation at 37° C. and 5% CO2, the cell culture supernatants were collected for multiplex assay analysis, while the adherent cells were processed for immunocytofluorescence.

We chose to immunostain CD31 and VE-Cadherin, because these proteins cluster together at the adherens junction in functional endothelial monolayers. CD31 is known to be truncated and miss the first, most membrane-distal, extracellular domains on stressed endothelial cells. Thus, to detect intact (functional) CD31 molecules we used the mouse monoclonal antibody JC70A (Dako, #M0823) as this antibody typically fails to stain cells that express a truncated CD31.

The staining protocol was the same as described in the previous paragraphs for adherent platelets, except that the permeabilization step was not performed, and that the following antibodies were used:
primary antibody: mouse anti-human CD31 (10 μg/mL, Dako M0823)
secondary antibody: goat anti-mouse IgG, A488 (5 μg/mL, Invitrogen A11029).

The measure of pro-inflammatory and pro-coagulant endothelial biomarkers in the cell culture supernatants was achieved using a custom multiplexed cytometric bead assay (Luminex technology). Each bead contains two different types of fluorophores. This makes it possible to detect several types of beads in the same well: each type of bead is identified by different fluorescence intensities at two emission wavelengths. Each type of capture antibody is associated with one type of bead. The quantity of captured antigen on each bead is measured by the fluorescence intensity of the phycoerythrin (PE) bound to detection antibodies. Thus, the concentration of several antigens can be determined in the same well.

In order to analyze the influence of “PDA” and “P8RI” coatings on the phenotype of cultivated endothelial cells, different types of soluble proteins produced by activated endothelial cells were quantified. A range of soluble molecules considered as markers of EC pro-inflammatory and pro-thrombotic phenotype were quantified in preliminary experiments.

IL-6, a pro-inflammatory cytokine involved in lymphocyte growth and differentiation, acute phase reaction and fever, and IL-8, a chemokine that induces granulocytes migration and phagocytosis, but also promotes angiogenesis, are both known to be released by endothelial cells in response to inflammatory stimuli, such as LPS and TNFα. They were therefore selected as markers of EC pro-inflammatory phenotype. CD62E and VCAM-1 (Vascular Cell Adhesion Molecule-1) are transmembrane glycoproteins of the CAM (cell adhesion molecules) family and are responsible for leukocyte adhesion on ECs. Thus, they are not primarily expressed as soluble molecules. However, cytokine activated endothelial cells are known not only to exhibit increased surface expression of CD62E and VCAM-1, but also to produce soluble forms of these proteins, as a result of unidentified cleavage of shedding processes. For this reason, CD62E and VCAM-1 were also chosen as markers of EC pro-inflammatory phenotype. The pro-thrombotic phenotype of the ECs was assessed by the quantification of PAI-1 (Plasminogen Activator Inhibitor-1), a serine protease inhibitor directed against tissue plasminogen activator (tPA) and urokinase (uPA). Since tPA and uPA activate plasminogen and are therefore the main initiators of fibrinolysis, the action of PAI-1 is pro-thrombotic. These five proteins were quantified in cell culture supernatants in several experiments, performed with cells from different donors. Each experiment included eight technical replicates.

The endothelial cell culture supernatants were centrifuged at high speed in order to remove any dead cell and debris, then they were transferred to polypropylene 96-well plates, sealed and stored at −80° C. until the day of the assay. They were then thawed and diluted in staining buffer. The optimal concentration was determined according to previous experiments. The diluted supernatants were transferred to a flat-bottom 96-well plate containing a mix of the capture beads. A standard range was added to the same plate by serial dilution of standard solutions (Bio-Rad), containing a known concentration of each antigen. The plate was incubated on an orbital shaker for 1 h, in order for the capture antibodies to bind their antigens. The plate was then rinsed with wash buffer on an automatic wash station, which rinses the wells while holding the magnetic beads at the bottom of the wells. The biotinylated detection antibodies, diluted according to the manufacturer's instructions, were then incubated with the beads for 30 minutes. Finally, after rinsing, streptavidin-PE was added to each well and incubated for 10 minutes, so that the streptavidin could bind to the detection antibody's biotin. After a last rinsing step, the beads were resuspended in assay buffer and analyzed on an assay reader (Bio-Plex 200, Bio-Rad). The statistical analysis of the results was performed with Kruskal-Wallis test. This nonparametric test was chosen as the number of replicates was too low to assess their Gaussian distribution, and hence to fulfill the conditions for an ANOVA test. Differences were considered significant for p<0.05.

The “PDA+P8RI” Coating Favors Rapid and Functional Endothelialization of CoCr Surfaces

In view of representative images of immunostained endothelial cells, the major difference between the three groups (CoCr, PDA and P8RI) is that the adhesion of endothelial cells appears much lesser on bare CoCr surfaces than on “PDA” and “P8RI” surfaces, since the ECs detached only from the bare surfaces. The “PDA” and “P8RI” coatings on experimental CoCr disks both allowed the formation of a functional confluent layer of endothelial cells, as evidenced by the distribution of CD31 at intercellular junctions.

The “PDA+P8RI” Coating Tends to Induce an Anti-Inflammatory and Anti-Thrombotic Phenotype in Endothelial Cells

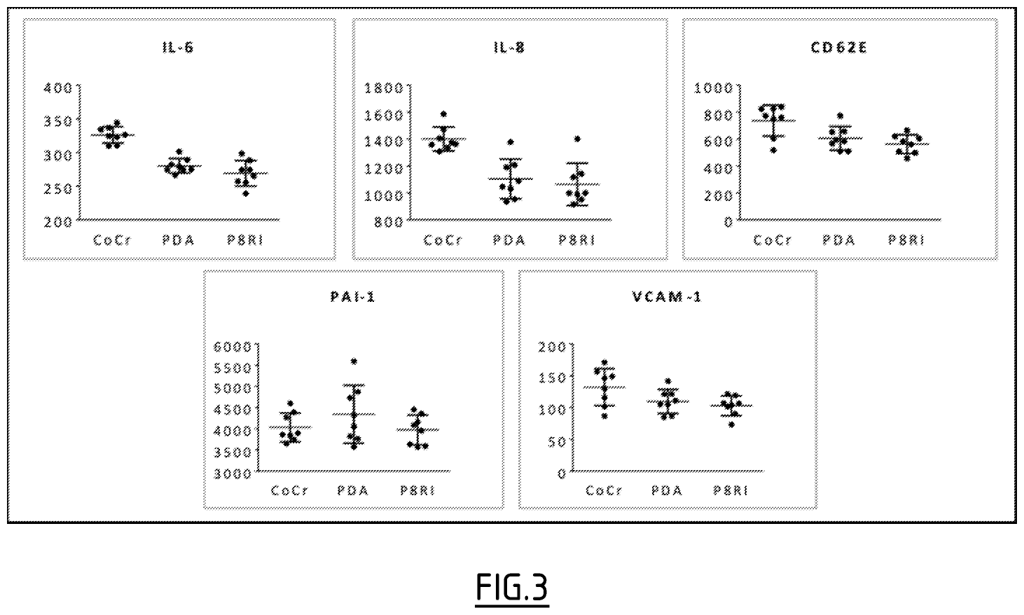

As can be seen on FIG. 3, the variability between the technical replicates of the multiplex assays was generally low, and clear differences between groups (cells cultivated on bare CoCr, “PDA” or “P8RI” surfaces) were found within one experiment. In particular, a tendency consistently appeared in IL-6, IL-8, CD62E and VCAM-1: concentrations on “PDA” were lower than on “CoCr”, and concentrations on “P8RI” lower than on “PDA”. This experiment thus suggested an anti-inflammatory effect of the PDA coating compared to bare CoCr, and a further effect of P8RI immobilization.

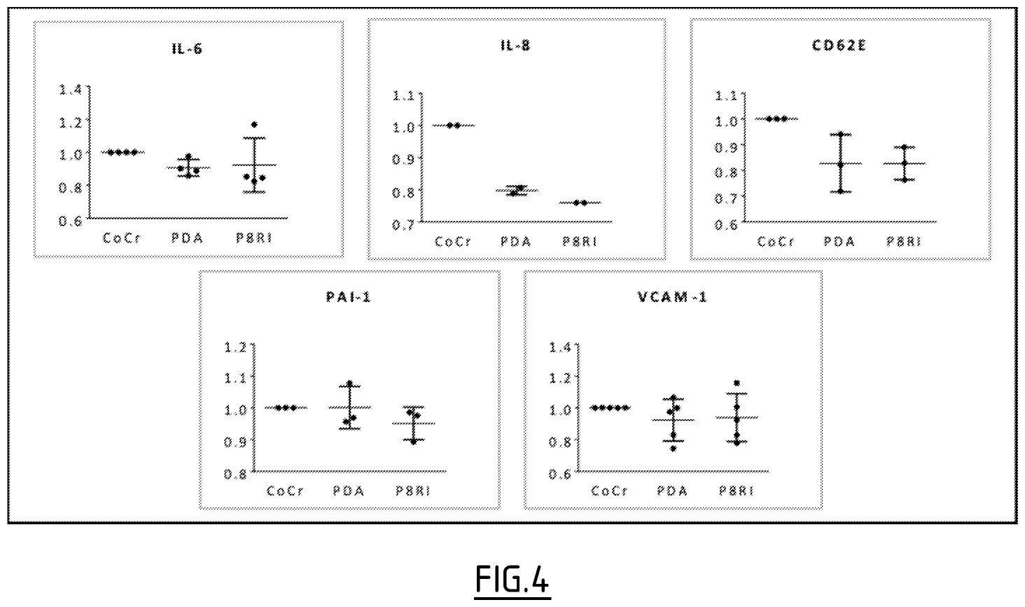

The synthesis of the results obtained from all cell culture supernatant analyses is presented on FIG. 4. The multiplex assay was performed on five biological replicates (endothelial cells from five different donors). Each point on the graphs of FIG. 4 represents the average of the technical replicates for one experiment. As in the experiment presented on FIG. 3, the concentrations of IL-6, IL-8, CD62E and VCAM-1 are lower in the “PDA” and “P8RI” groups than in the “CoCr” group. An additional effect of P8RI immobilization is observed on IL-8, but also on PAI-1 concentrations, suggesting a slight anti-thrombotic effect of the P8RI coating. However, given the low number of replicates and high variability between experiments, the differences did not reach the statistical significance according to the Kruskal-Wallis test.

Therefore, the apparent anti-thrombotic and anti-inflammatory effects of “PDA” and “P8RI” coatings can only be expressed in terms of tendency.

In Vivo Evaluation of the Biocompatibility of P8RI-Coated Surfaces

Bare and “PDA+P8RI”-coated FDSs were implanted in animal models in order to assess the effect of the coating on the stents' in vivo environment. Medium animals (rabbits) were used for the implantation of FDSs, since the arteries of these animals are large enough to be compatible with the size of stents designed for human use.

The coating strategy which was developed has been applied to commercially available stents. The biocompatibility of the “PDA+P8RI” coating could hence be evaluated also in vivo, by comparing the performance of the “PDA+P8RI”-coated stents with that of the parent bare metal or active device, made of the same alloy. The Multilink BMS, made of CoCr, was suitable for both P8RI immobilization strategies developed during this study (the PDA-P8RI coating and the direct immobilization of P8RI on plasma-functionalized CoCr), whereas only the PDA-based strategy could be applied to the nitinol Silk FDS. Thus, commercially available nitinol FDSs (Silk) were compared only to “PDA-P8RI” FDSs.

P8RI-coated and control FDSs were implanted in the right carotid arteries of rabbits, after creation of elastase-induced saccular aneurysms. Due to the size of the target arteries in these animals, which are large enough to be compatible with the size of stents designed for human use, these models are widely used for the issue of in vivo stent biocompatibility.

Implantation of Flow Diverting Stents in a Rabbit Elastase Aneurysm Model

In order to assess the effect of the “PDA+P8RI” coating on the performances of FDSs in terms of aneurysm occlusion and integration at the blood/vessel interface, the inventors conducted in vivo experiments with bare and P8RI-coated FDSs, in a rabbit elastase aneurysm model.

The FDSs used in this study were Silk stents, supplied by the manufacturer, Balt. P8RI-coated Silk FDS stents were prepared following the procedure as described above and sterilized by beta radiations prior to their implantation in vivo.

The rabbit elastase aneurysm is one of the most commonly used models for the testing of FDSs. It consists in creating an artificial aneurysm in the proximal segment of the right common carotid artery, by incubating elastase in the ligated artery for 20 minutes, so that this protease can hydrolyze elastin in the artery wall and promote its dilative remodeling. The aneurysm then grows over the course of a few weeks. Despite its extracranial location, this procedure results in a stable aneurysm with hemodynamic, morphological and histological features similar to those of human intracranial aneurysms.

The elastase aneurysm model was run in three separate sets of experiments. The first two were run in the absence of anti-platelet treatment. The presence of a healing thrombus in the stented arteries of these series prevented the analysis of the luminal endothelialization. The inventors therefore performed a third series of experiments for which we created the model in 8 male New Zealand rabbits. Three weeks after the creation of the aneurysms, unmodified (n=4) and P8RI-coated (N=4) FDSs were implanted in the native right subclavian artery of each animal, so as to occlude the experimental saccular aneurysm. The SEM analysis was not suitable for the evaluation of the biocompatibility of the device in this model, due to the asymmetric positioning of the saccular aneurysm which prevented its longitudinal opening and observation on the luminal side. Thus, in two rabbits, an additional FDS was implanted further in the contralateral subclavian artery, in order to assess the integration of the devices in the vessel wall by scanning electron microscopy (SEM) observation of its luminal side.

The animals were administered 75 mg of aspirin daily and terminated 4 weeks after FDS implantation. This duration was chosen because it had been shown that the inflammatory reaction in aneurysms created with this model is stopped 4 weeks after endovascular treatment, thus resulting in an aneurysm filled with acellular connective tissue, resembling the stable aneurysms seen in humans. Immediately after the sacrifice of each animal, the subclavian artery segment containing the FDS was explanted, together with the aneurysm. The explanted arteries were then processed for histology. After paraformaldehyde fixation, they were dehydrated by successive incubations in ethanol baths of increasing concentration. Then, they were impregnated with a methyl methacrylate solution. Finally, the polymerization of the methyl methacrylate monomers was triggered by the addition of benzoyl peroxide and N, N-dimethyl-p-toluidine.

This process resulted in the embedding of the explanted arteries in a poly(methyl methacrylate) (PMMA) resin. Although it is more complex than the usual histology techniques (paraffin embedding and cryosection), PMMA embedding is necessary for the sectioning of soft tissues, such as arteries, containing hard materials, like metallic stents. Indeed, the PMMA resin greatly decreases the difference in hardness between the stent struts and the surrounding tissue, which makes it possible to section the sample into thin slices without tearing off the tissue.

The embedded arteries were then sectioned transversally in a microtome equipped with a tungsten carbide blade, which is harder than the steel blade used for the sectioning of usual paraffin-embedded tissues. The resulting histology slides were stained by Masson's trichrome, which colors the connective tissues in blue, the cytoplasm in pink and the nuclei in purple. In order to better distinguish the smooth muscle cells, alpha-smooth muscle actin (aSMA) was stained by immunofluorescence in serial sections.

The two extra FDSs which had been implanted in the subclavian arteries of the rabbits, distally from the aneurysm, were processed for SEM observation, as detailed in the previous section.

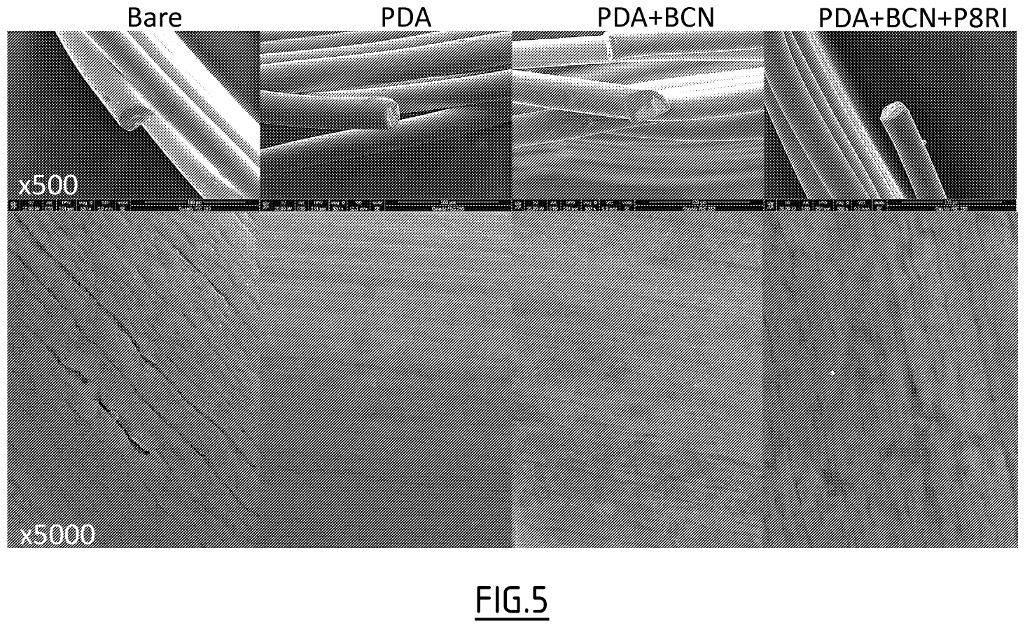

FIG. 5 shows SEM images of a P8RI-coated stent implanted in the contralateral, untouched subclavian artery of a rabbit, remotely from the aneurysm. Over its 4 week-period of implantation, the FDS has been entirely covered by neointimal growth, which yields the appearance of a velvet cover over the stent struts. A few adherent cells, identified as non-activated leukocytes because of their round shape, can be seen at the luminal surface of the vessel.

Transversal resin sections of implanted bare and P8RI-coated stents were stained by Masson's trichrome and immunofluorescence. The struts of both types of stents have been covered by neointimal formation over the entire circumference of the vessel. However, the characteristics of the tissue that covers the stent struts strikingly differ with the type of stents. On the P8RI-coated stent the struts are way back in the arterial wall, covered by a thick, organized neointima, with layers of SMCs and oriented sheets of ECM, and exhibit a continuous endothelial monolayer. This organization is seen both in front of the aneurysmal neck and away from it. Contrastingly, on the bare stent, the struts are barely covered by a thin neointima which appears poorly organized, devoid of SMCs and covered with a discontinuous endothelium. The better organization and the presence of SMCs and a continuous endothelium on the neointima that covers the P8RI-coated stent constitute promising results, since the impermeability of the neointima covering the stent structure, especially in front of the aneurysmal neck, is key to successful aneurysm occlusion and stable arterial healing. Besides, the media of the aneurysmal wall also appears less organized in the animal treated with a bare FDS, since it does not exhibit the layers of oriented SMCs seen in the other animal. Moreover, red blood cells and infiltrated leukocytes are visible in the aneurysmal wall of the animal treated with a bare FDS. Their presence indicates that neoangiogenesis and inflammation take place in the aneurysm wall, making it more susceptible to rupture. These phenomena are not seen in the aneurysmal wall of the animal treated with a “PDA-P8RI”-coated FDS.

1. A method for the modification of a surface of a metallic implantable device for interventional neuroradiology by grafting a CD31-derived peptide onto the surface of said device, wherein the CD31-derived peptide consists of a sequence selected from the group consisting of: SEQ ID NO: 2 to 8, SEQ ID NO: 12 to 79, and SEQ ID NO: 81, said method comprising the following steps:
a) coating a polydopamine layer onto the surface of a metallic implantable device for interventional neuroradiology in order to obtain a polydopamine coated surface;
b) modifying the polydopamine coated surface by the addition of a linker, comprising at least one reactive moiety chosen from alkyne functions, in order to obtain a modified polydopamine coated surface; and
c) adding a CD31-derived peptide comprising an azide terminal group and reacting the CD31-derived peptide comprising an azide terminal group with the alkyne function of the linker of step b), in order to obtain a polydopamine coated surface grafted by a CD31-derived peptide, wherein the CD31-derived peptide comprising an azide terminal group is a CD31-derived peptide as defined above which is chemically modified with an azide terminal group.

2. The method of claim 1, wherein step a) comprises contacting the surface of the device with a solution of dopamine and incubating said device and said solution.

3. The method of claim 1, wherein step a) is followed by a rinsing step of the polydopamine coated surface.

4. The method of claim 1, wherein the polydopamine layer has a thickness comprised between 20 nm and 100 nm.

5. The method of claim 1, wherein the linker of step b) has the following formula (I-1): 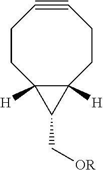
wherein R is a radical of formula —X₁-A₁-NH₂,
X₁ being chosen from the group consisting of: —CONH—, —CO—, —CS— and —CSNH, and
A₁ being an alkylene radical comprising from 2 to 40 carbon atoms, possibly interrupted by at least one oxygen atom.

6. The method of claim 1, wherein the linker of step b) has the following formula (I): 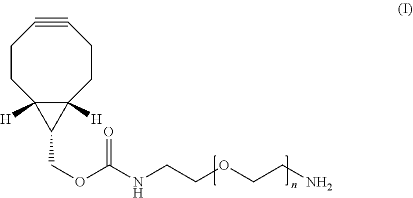
wherein n is an integer comprised between 2 and 14.

7. The method of claim 1, wherein the linker of step b) has the following formula:

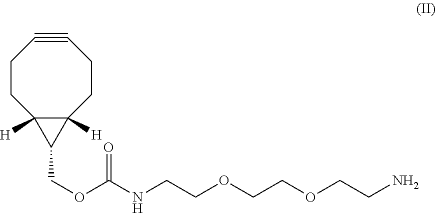 [SMILES: NCCOCCOCCNC(=O)OC[C@@H]1[C@@H]2CCC#CCC[C@@H]21]

8. The method of claim 1, wherein step b) comprises contacting the polydopamine coated surface of the device with a solution of the linker and incubating said device and said solution, under stirring.

9. The method of claim 8, wherein step b) is followed by a rinsing step of the modified polydopamine coated surface.

10. The method of claim 1, wherein the layer made of the linker has a thickness comprised between 0.03 nm and 3 nm.

11. The method of claim 1, wherein the CD31-derived peptide comprising an azide terminal group has the formula:
N⁻═N⁺═N—(CD31-derived peptide)-OH
wherein the CD31-derived peptide consists of a sequence selected from the group consisting of: SEQ ID NO: 2 to 8, SEQ ID NO: 12 to 79, and SEQ ID NO: 81.

12. The method of claim 1, wherein the CD31-derived peptide comprising an azide terminal group comprises a peptide having the sequence KWPALFVR (SEQ ID NO: 6) and consisting of D-enantiomer amino acids.

13. The method of claim 1, wherein the CD31-derived peptide comprising an azide terminal group comprises a peptide having the sequence KGGGKWPALFVR (SEQ ID NO: 81) and consisting of D-enantiomer amino acids.

14. The method of claim 1, wherein the CD31-derived peptide comprising an azide terminal group has the following formula:

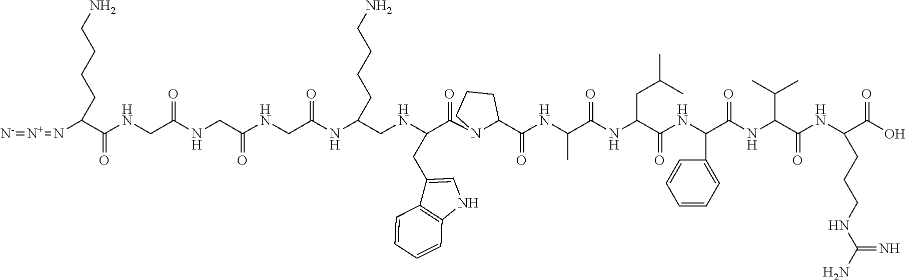 [SMILES: CC(C)CC(NC(=O)C(C)NC(=O)C1CCCN1C(=O)C(Cc1c[nH]c2ccccc12)NCC(CCCCN)NC(=O)CNC(=O)CNC(=O)CNC(=O)C(CCCCN)N=[N+]=[N-])C(=O)NC(C(=O)NC(C(=O)NC(CCCNC(=N)N)C(=O)O)C(C)C)c1ccccc1]

15. The method of claim 1, wherein step c) comprises a step of copper-free click chemistry reaction.

16. The method of claim 1, wherein the thickness of the layers made of the linker and of the CD31-derived peptide is comprised between 0.5 nm and 15 nm.

17. The method of claim 1, wherein the thickness of the polydopamine layer and of the layers made of the linker and of the CD31-derived peptide is comprised between 20 nm and 200 nm.

18. The method of claim 1, wherein the surface of the device is made of metals or metal alloys.

19. The method of claim 1, wherein the device for interventional neuroradiology is chosen from the group consisting of: intracranial stents, flow-diverter stents, and metallic embolization devices.

20. A modified surface device for interventional neuroradiology, wherein the surface of said device is grafted by a CD31-derived peptide, said device and CD31-derived peptide being as defined in claim 1, said modified surface device being obtainable by the method of claim 1.

21. The modified surface device of claim 20, wherein said modified surface comprises a coating made of a layer of polydopamine and a layer made of the linker and the CD31-derived peptide.


SEQUENCE LISTING (81 sequences)

SEQ ID NO: 1 - protein, 738 aa, Homo sapiens
  SIGNAL (1)..(27)
  DOMAIN (28)..(601): Extracellular domain
  DOMAIN (34)..(121): First Ig-like domain
  DOMAIN (145)..(233): Second Ig-like domain
  DOMAIN (236)..(315): Third Ig-like domain
  DOMAIN (328)..(401): Fourth Ig-like domain
  DOMAIN (424)..(493): Fifth Ig-like domain
  DOMAIN (499)..(591): Sixth Ig-like domain
  DOMAIN (592)..(601): Juxta-membrane domain
  DOMAIN (602)..(620): Transmembrane domain
  DOMAIN (621)..(738): Cytoplasmic domain
MQPRWAQGATMWLGVLLTLLLCSSLEGQENSFTINSVDMKSLPDWTVQNGKNLTLQCFADVSTTSHVKPQHQMLFYKDDVLFYNISSMKSTESYFIPEVRIYDSGTYKCTVIVNNKEKTTAEYQLLVEGVPSPRVTLDKKEAIQGGIVRVNCSVPEEKAPIHFTIEKLELNEKMVKLKREKNSRDQNFVILEFPVEEQDRVLSFRCQARIISGIHMQTSESTKSELVTVTESFSTPKFHISPTGMIMEGAQLHIKCTIQVTHLAQEFPEIIIQKDKAIVAHNRHGNKAVYSVMAMVEHSGNYTCKVESSRISKVSSIVVNITELFSKPELESSFTHLDQGERLNLSCSIPGAPPANFTIQKEDTIVSQTQDFTKIASKSDSGTYICTAGIDKVVKKSNTVQIVVCEMLSQPRISYDAQFEVIKGQTIEVRCESISGTLPISYQLLKTSKVLENSTKNSNDPAVFKDNPTEDVEYQCVADNCHSHAKMLSEVLRVKVIAPVDEVQISILSSKVVESGEDIVLQCAVNEGSGPITYKFYREKEGKPFYQMTSNATQAFWTKQKASKEQEGEYYCTAFNRANHASSVPRSKILTVRVILAPWKKGLIAVVIIGVIIALLIIAAKCYFLRKAKAKQMPVEMSRPAVPLLNSNNEKMSDPNMEANSHYGHNDDVRNHAMKPINDNKEPLNSDVQYTEVQVSSAESHKDLGKKDTETVYSEVRKAVPDAVESRYSRTEGSLDGT

SEQ ID NO: 2 - protein, 6 aa, Artificial Sequence
  mouse or human-derived CD31 peptide
LAPWKK

SEQ ID NO: 3 - protein, 10 aa, Artificial Sequence
  mouse-derived CD31 peptide
VRVFLAPWKK

SEQ ID NO: 4 - protein, 10 aa, Artificial Sequence
  human-derived CD31 peptide
VRVILAPWKK

SEQ ID NO: 5 - protein, 8 aa, Artificial Sequence
  murine-derived CD31 peptide
RVFLAPWK

SEQ ID NO: 6 - protein, 8 aa, Artificial Sequence
  murine-derived CD31 peptide
KWPALFVR

SEQ ID NO: 7 - protein, 8 aa, Artificial Sequence
  human-derived CD31 peptide
RVILAPWK

SEQ ID NO: 8 - protein, 8 aa, Artificial Sequence
  human-derived CD31 sequence
KWPALIVR

SEQ ID NO: 9 - protein, 727 aa, Mus musculus
MLLALGLTLVLYASLQAEENSFTINSIHMESLPSWEVMNGQQLTLECLVDISTTSKSRSQHRVLFYKDDAMVYNVTSREHTESYVIPQARVFHSGKYKCTVMLNNKEKTTIEYEVKVHGVSKPKVTLDKKEVTEGGVVTVNCSLQEEKPPIFFKIEKLEVGTKFVKRRIDKTSNENFVLMEFPIEAQDHVLVFRCQAGILSGFKLQESEPIRSEYVTVQESFSTPKFEIKPPGMIIEGDQLHIRCIVQVTHLVQEFTEIIIQKDKAIVATSKQSSEAVYSVMAMVEYSGHYTCKVESNRISKASSIMVNITELFPKPKLEFSSSRLDQGELLDLSCSVSGTPVANFTIQKEETVLSQYQNFSKIAEESDSGEYSCTAGIGKVVKRSGLVPIQVCEMLSKPSIFHDAKSEIIKGHAIGISCQSENGTAPITYHLMKAKSDFQTLEVTSNDPATFTDKPTRDMEYQCRADNCHSHPAVFSEILRVRVIAPVDEVVISILSSNEVQSGSEMVLRCSVKEGTSPITFQFYKEKEDRPFHQAVVNDTQAFWHNKQASKKQEGQYYCTASNRASSMRTSPRSSTLAVRVFLAPWKKGLIAVVVIGVVIATLIVAAKCYFLRKAKAKQKPVEMSRPAAPLLNSNSEKISEPSVEANSHYGYDDVSGNDAVKPINQNKDPQNMDVEYTEVEVSSLEPHQALGTRATETVYSEIRKVDPNLMENRYSRTEGSLNGT

SEQ ID NO: 10 - protein, 739 aa, Bos taurus
MQLRWTQRGMMWLGALLTLLLCSSLKGQENSFTINSIHMQILPHSTVQNGENLTLQCLVDVSTTSRVKPLHQVLFYKDDVLLHNVSSRRNTESYLIPHVRVCDSGRYKCNVILNNKEKTTPEYEVWVKGVSDPRVTLDKKEVIEGGVVVVNCSVPEEKAPVHFTIEKFELNIRGAKKKREKTSQNQNFVTLEFTVEEQDRTIRFQCQAKIFSGSNVESSRPIQSDLVTVRESFSNPKFHIIPEGKVMEGDDLQVKCTVQVTHQAQSFPEIIIQKDREIVAHNSLSSEAVYSVMATTEHNGNYTCKVEASRISKVSSVVVNVTELFSKPKLESSATHLDQGEDLNLLCSIPGAPPANFTIQKGGMTVSQTQNFTKRVSEWDSGLYTCVAGVGRVFKRSNTVQITVCEMLSKPSIFHDSRSEVIKGQTIEVSCQSVNGTAPIFYQLSNTSKPVANQSVGSNKPAIFRVKPTKDVEYCCSADNCHSHSKMFSEVLRVKVIAPVDEAQLVVLKGEVEPGEPIVFYCSVNEGSFPITYKFYKEKESKPFYQDTINATQIMWHKTTASKEYEGQYYCTASNRANLSKHVIQSNTLTVRVYLPLEKGLIAVVVIGVIIVTLVLGAKCYFLKKAKAKQMPVEMSRPAVPLLNSNNEKTLSDAGTEADRHYGYNEDVGNHAMKPLNENKEPLTLDVEYTEVEVTSPEPHQGLGTKGTETETVYSEIRKADPDFVENRYSRTEGSLDGS

SEQ ID NO: 11 - protein, 740 aa, Sus scrofa
MRLRWTQGGNMWLGVLLTLQLCSSLEGQENSFTINSIHMEMLPGQEVHNGENLTLQCIVDVSTTSSVKPQHQVLFYKDDVLFHNVSSTKNTESYFISEARVYNSGRYKCTVILNNKEKTTAEYKVVVEGVSNPRVTLDKKEVIEGGVVKVTCSVPEEKPPVHFIIEKFELNVRDVKQRREKTANNQNSVTLEFTVEEQDRVILFSCQANVIFGTRVEISDSVRSDLVTVRESFSNPKFHISPKGVIIEGDQLLIKCTIQVTHQAQSFPEIIIQKDKEIVAHSRNGSEAVYSVMATVEHNSNYTCKVEASRISKVSSIMVNITELFSRPKLKSSATRLDQGESLRLWCSIPGAPPEANFTIQKGGMMMLQDQNLTKVASERDSGTYTCVAGIGKVVKRSNEVQIAVCEMLSKPSIFHDSGSEVIKGQTIEVSCQSINGTSPISYQLLKGSDLLASQNVSSNEPAVFKDNPTKDVEYQCIADNCHSHAGMPSKVLRVKVIAPVEEVKLSILLSEEVESGQAIVLQCSVKEGSGPITYKFYKEKENKPFHQVTLNDTQAIWHKPKASKDQEGQYYCLASNRATPSKNFLQSNILAVRVYLAPWKKGLIAVVVIAVIIAVLLLGARFYFLKKSKAKQMPVEMCRPAAPLLNSNNEKTLSDPNTEANRHYGYNEDVGNHAMKPLNENKEPLTLDVEYTEVEVTSPEPHRGLGTKGTETVYSEIRKADPDLVENRYSRTEGSLDGT

SEQ ID NO: 12 - protein, 23 aa, Artificial Sequence
  human-derived CD31 peptide
NHASSVPRSKILTVRVILAPWKK

SEQ ID NO: 13 - protein, 15 aa, Artificial Sequence
  peptide
SSTLAVRVFLAPWKK

SEQ ID NO: 14 - protein, 14 aa, Artificial Sequence
  peptide
STLAVRVFLAPWKK

SEQ ID NO: 15 - protein, 13 aa, Artificial Sequence
  peptide
TLAVRVFLAPWKK

SEQ ID NO: 16 - protein, 12 aa, Artificial Sequence
  peptide
LAVRVFLAPWKK

SEQ ID NO: 17 - protein, 11 aa, Artificial Sequence
  peptide
AVRVFLAPWKK

SEQ ID NO: 18 - protein, 9 aa, Artificial Sequence
  peptide
RVFLAPWKK

SEQ ID NO: 19 - protein, 8 aa, Artificial Sequence
  peptide
VFLAPWKK

SEQ ID NO: 20 - protein, 7 aa, Artificial Sequence
  peptide
FLAPWKK

SEQ ID NO: 21 - protein, 5 aa, Artificial Sequence
  peptide
APWKK

SEQ ID NO: 22 - protein, 4 aa, Artificial Sequence
  peptide
PWKK

SEQ ID NO: 23 - protein, 15 aa, Artificial Sequence
  peptide
SKILTVRVILAPWKK

SEQ ID NO: 24 - protein, 14 aa, Artificial Sequence
  peptide
KILTVRVILAPWKK

SEQ ID NO: 25 - protein, 13 aa, Artificial Sequence
  peptide
ILTVRVILAPWKK

SEQ ID NO: 26 - protein, 12 aa, Artificial Sequence
  peptide
LTVRVILAPWKK

SEQ ID NO: 27 - protein, 11 aa, Artificial Sequence
  peptide
TVRVILAPWKK

SEQ ID NO: 28 - protein, 9 aa, Artificial Sequence
  peptide
RVILAPWKK

SEQ ID NO: 29 - protein, 8 aa, Artificial Sequence
  peptide
VILAPWKK

SEQ ID NO: 30 - protein, 7 aa, Artificial Sequence
  peptide
ILAPWKK

SEQ ID NO: 31 - protein, 15 aa, Artificial Sequence
  peptide
SSMRTSPRSSTLAVR

SEQ ID NO: 32 - protein, 14 aa, Artificial Sequence
  peptide
SSMRTSPRSSTLAV

SEQ ID NO: 33 - protein, 13 aa, Artificial Sequence
  peptide
SSMRTSPRSSTLA

SEQ ID NO: 34 - protein, 12 aa, Artificial Sequence
  peptide
SSMRTSPRSSTL

SEQ ID NO: 35 - protein, 11 aa, Artificial Sequence
  peptide
SSMRTSPRSST

SEQ ID NO: 36 - protein, 10 aa, Artificial Sequence
  peptide
SSMRTSPRSS

SEQ ID NO: 37 - protein, 9 aa, Artificial Sequence
  peptide
SSMRTSPRS

SEQ ID NO: 38 - protein, 8 aa, Artificial Sequence
  peptide
SSMRTSPR

SEQ ID NO: 39 - protein, 7 aa, Artificial Sequence
  peptide
SSMRTSP

SEQ ID NO: 40 - protein, 6 aa, Artificial Sequence
  peptide
SSMRTS

SEQ ID NO: 41 - protein, 5 aa, Artificial Sequence
  peptide
SSMRT

SEQ ID NO: 42 - protein, 4 aa, Artificial Sequence
  peptide
SSMR

SEQ ID NO: 43 - protein, 15 aa, Artificial Sequence
  peptide
NHASSVPRSKILTVR

SEQ ID NO: 44 - protein, 14 aa, Artificial Sequence
  peptide
NHASSVPRSKILTV

SEQ ID NO: 45 - protein, 13 aa, Artificial Sequence
  peptide
NHASSVPRSKILT

SEQ ID NO: 46 - protein, 12 aa, Artificial Sequence
  peptide
NHASSVPRSKIL

SEQ ID NO: 47 - protein, 11 aa, Artificial Sequence
  peptide
NHASSVPRSKI

SEQ ID NO: 48 - protein, 10 aa, Artificial Sequence
  peptide
NHASSVPRSK

SEQ ID NO: 49 - protein, 9 aa, Artificial Sequence
  peptide
NHASSVPRS

SEQ ID NO: 50 - protein, 8 aa, Artificial Sequence
  peptide
NHASSVPR

SEQ ID NO: 51 - protein, 7 aa, Artificial Sequence
  peptide
NHASSVP

SEQ ID NO: 52 - protein, 6 aa, Artificial Sequence
  peptide
NHASSV

SEQ ID NO: 53 - protein, 5 aa, Artificial Sequence
  peptide
NHASS

SEQ ID NO: 54 - protein, 4 aa, Artificial Sequence
  peptide
NHAS

SEQ ID NO: 55 - protein, 15 aa, Artificial Sequence
  peptide
TSPRSSTLAVRVFLA

SEQ ID NO: 56 - protein, 13 aa, Artificial Sequence
  peptide
SPRSSTLAVRVFL

SEQ ID NO: 57 - protein, 11 aa, Artificial Sequence
  peptide
PRSSTLAVRVF

SEQ ID NO: 58 - protein, 9 aa, Artificial Sequence
  peptide
RSSTLAVRV

SEQ ID NO: 59 - protein, 7 aa, Artificial Sequence
  peptide
SSTLAVR

SEQ ID NO: 60 - protein, 5 aa, Artificial Sequence
  peptide
STLAV

SEQ ID NO: 61 - protein, 15 aa, Artificial Sequence
  peptide
SVPRSKILTVRVILA

SEQ ID NO: 62 - protein, 13 aa, Artificial Sequence
  peptide
VPRSKILTVRVIL

SEQ ID NO: 63 - protein, 11 aa, Artificial Sequence
  peptide
PRSKILTVRVI

SEQ ID NO: 64 - protein, 9 aa, Artificial Sequence
  peptide
RSKILTVRV

SEQ ID NO: 65 - protein, 7 aa, Artificial Sequence
  peptide
SKILTVR

SEQ ID NO: 66 - protein, 5 aa, Artificial Sequence
  peptide
KILTV

SEQ ID NO: 67 - protein, 4 aa, Artificial Sequence
  peptide
RVFL

SEQ ID NO: 68 - protein, 5 aa, Artificial Sequence
  peptide
RVFLA

SEQ ID NO: 69 - protein, 6 aa, Artificial Sequence
  peptide
RVFLAP

SEQ ID NO: 70 - protein, 7 aa, Artificial Sequence
  peptide
RVFLAPW

SEQ ID NO: 71 - protein, 4 aa, Artificial Sequence
  peptide
RVIL

SEQ ID NO: 72 - protein, 5 aa, Artificial Sequence
  peptide
RVILA

SEQ ID NO: 73 - protein, 6 aa, Artificial Sequence
  peptide
RVILAP

SEQ ID NO: 74 - protein, 7 aa, Artificial Sequence
  peptide
RVILAPW

SEQ ID NO: 75 - protein, 15 aa, Artificial Sequence
  peptide
RSKILTVRVILAPWK

SEQ ID NO: 76 - protein, 14 aa, Artificial Sequence
  peptide
SKILTVRVILAPWK

SEQ ID NO: 77 - protein, 13 aa, Artificial Sequence
  peptide
KILTVRVILAPWK

SEQ ID NO: 78 - protein, 12 aa, Artificial Sequence
  peptide
ILTVRVILAPWK

SEQ ID NO: 79 - protein, 11 aa, Artificial Sequence
  peptide
LTVRVILAPWK

SEQ ID NO: 80 - protein, 4 aa, Artificial Sequence
  spacer
KGGG

SEQ ID NO: 81 - protein, 12 aa, Artificial sequence
  spacer + peptide
KGGGKWPALFVR